(19) United States
(12) Patent Application Publication
(10) Pub. No.: US 2014/0031372 A1
(43) Pub. Date: Jan. 30, 2014

(54) CICLETANINE IN COMBINATION WITH ORAL ANTIDIABETIC AND/OR BLOOD LIPID-LOWERING AGENTS AS A COMBINATION THERAPY FOR DIABETES AND METABOLIC SYNDROME

(71) Applicant: CoTherix, Inc., (unknown)

(72) Inventors: Benson M. Fong, San Francisco, CA (US); Glenn V. Cornett, Palo Alto, CA (US)

(73) Assignee: CoTherix, Inc.

(21) Appl. No.: 14/044,675

(22) Filed: Oct. 2, 2013

Related U.S. Application Data
(63) Continuation of application No. 13/352,067, filed on Jan. 17, 2012.
Application No. 13/352,067 is a continuation of application No. 12/837,222, filed on Jul. 15, 2010, now abandoned.
Application No. 12/837,222 is a continuation of application No. 10/929,108, filed on Aug. 27, 2004, now abandoned.
(60) Provisional application No. 60/498,916, filed on Aug. 29, 2003.

(51) Int. Cl. A61K 31/4355; A61K 45/06

(52) U.S. Cl. CPC A61K 31/4355; A61K 45/06
USPC 514/255.06; 514/302

(57) ABSTRACT

Preferred embodiments of the present invention are related to novel therapeutic drug combinations and methods for treating and/or preventing complications in patients with diabetes and/or metabolic syndrome. More particularly, aspects of the present invention are related to using a combination of cicletanine and an oral antidiabetic agent for treating and/or preventing complications (including microalbuminuria, nephropathies, retinopathies and other complications) in patients with diabetes or metabolic syndrome.

### RELATED APPLICATIONS

This application claims the benefit of UA Provisional Patent Application No. 60/498,916 filed Aug. 29, 2003, which is expressly incorporated herein by reference in its entirety.

### FIELD OF THE INVENTION

Preferred embodiments of the present invention are related to using a combination of cicletanine and an oral antidiabetic agent and/or a blood-lipid-lowering agent for treating and/or preventing complications (including microalbuminuria, nephropathies, retinopathies and other complications) in patients with diabetes or metabolic syndrome, for controlling blood glucose; and a combination of cicletanine and a lipid-lowering agent for controlling blood lipids and treating metabolic syndrome.

### BACKGROUND OF THE INVENTION

Diabetes is a chronic metabolic disorder which afflicts 14 million people in the United States, over two million of whom have its most severe form, childhood diabetes (also called juvenile, Type I or insulin-dependent diabetes). Type II Diabetes (DM II) makes up more than 85-90% of all diabetics, and is likely to be the next epidemic.

Patients with diabetes of all types have considerable morbidity and mortality from microvascular (retinopathy, neuropathy, nephropathy) and macrovascular (heart attacks, stroke, peripheral vascular disease) pathology, all of which carry an enormous cost. For example: a) Proliferative retinopathy (the leading cause of blindness for people under 65 years of age in the United States) and/or macular edema occur in about 50% of patients with type 2 diabetes, as do peripheral and/or autonomic neuropathy. b) The incidence of diabetic renal disease is 10% to 50% depending on ethnicity. c) Diabetics have heart attacks, strokes and peripheral vascular disease at about triple the rate of non-diabetics. The cost of treating diabetes and its complications exceeds $100 billion annually.

Non-insulin dependent diabetes mellitus develops especially in subjects with insulin resistance and a cluster of cardiovascular risk factors such as obesity, hypertension and dyslipidemia, a syndrome which first recently has been recognized and is named “The metabolic syndrome” (Alberti K. G., & Zimmet P. Z. 1998 Diabet Med 7:539-53).

In accordance with the WHO definition, a patient has metabolic syndrome if insulin resistance and/or glucose intolerance is present together with two or more of the following conditions: 1) reduced glucose tolerance or diabetes; 2) insulin sensitivity (under hyperinsulinemic, euglycemic conditions corresponding to a glucose uptake below the lower quartile for the background population); 3) increased blood pressure (≧140/90 mmHg); 4) increased plasma triglyceride (≧1.7 mmol/l) and/or low HDL cholesterol (<0.9 mmol/l for men; <1.0 mmol/l for women); 5) central adipositas (waist/hip ratio for men: >0.90 and for women >0.85) and/or Body Mass Index >30 kg/m²); 6) micro albuminuria (urine albumin excretion: ≧20 μg min⁻¹ or albumin/creatinine ratio ≧2.0 mg/mmol.

In the chronological sequence of impaired glucose tolerance, followed by early and late phases of type 2 diabetes, it is essential to start early with nonpharmacologic therapy, including physical activity, diet, and weight reduction. In addition, to reduce the incidence of macrovascular complications of diabetes, pharmacotherapy for disturbances in lipid metabolism and for hypertension is warranted (Goldberg, R. et al. 1998 Circulation 98:2513-2519; Pyorala, K. et al. 1997 Diabetes Care 20:614-620). Therefore, it has become increasingly evident that the treatment should aim at simultaneously normalizing blood glucose, blood pressure, lipids and body weight to reduce the morbidity and mortality. Unfortunately, until today no single drug that simultaneously attacks hyperglycemia, hypertension and dyslipidemia is available for patients with metabolic syndrome.

In general, there are three pharmacotherapeutic approaches typically relevant to the management of metabolic syndrome (insulin resistance syndrome, syndrome X):

1) Hypoglycemic agents: A) Oral antidiabetics (OADs); B) Insulin;

2) Antihypertensive agents;

3) Lipid-lowering agents.

Drug toxicity is an important consideration in the treatment of humans and animals. Toxic side effects resulting from the administration of drugs include a variety of conditions that range from low-grade fever to death. Drug therapy is justified only when the benefits of the treatment protocol outweigh the potential risks associated with the treatment. The factors balanced by the practitioner include the qualitative and quantitative impact of the drug to be used as well as the resulting outcome if the drug is not provided to the individual. Other factors considered include the physical condition of the patient, the disease stage and its history of progression, and any known adverse effects associated with a drug.

It is known that, for example, sulfonylureas can cause severe and lifethreatening hypoglycemia, due to their continuous action as long as they are present in the blood (Holman, R. R. & Turner, R. C., 1991 In: Textbook of Diabetes, Pickup, J. C., Williams, G., Eds; Blackwell Scientific Publ. London, pp. 462-476). Such an action may affect the myocytes in the heart increasing the risk of cardiac arrhythmias. On the other hand, metformin is known to cause stomach-malfunction and toxicity which can cause death by excessive dose of administration to a patient for a prolonged time (Innerfield, R. J. 1996 New Engl J Med 334:1611-1613). Glitazones (e.g., Actos®, Avandia®, Rezulin®; also known as the thiazolidinediones) tend to increase lipids. Troglitazone is known to have side effects, such as anemia, nausea, and hepatic toxicity (Eung-Jin Lee et al. 1998 Diabetes Science, Korea Medicine, 345-359; Ishii, S. et al. 1996 Diabetes 45: (Suppl. 2), 141A (abstracts) Watking, P. B. et al. 1998 N Engl J Med 338:916-917). Other reported adverse events include dyspnea, headache, thirst, gastrointestinal distress, insomnia, dizziness, incoordination, confusion, fatigue, pruritus, rash, alterations in blood cell counts, changes in serum lipids, acute renal insufficiency, and dryness of the mouth. Additional symptoms that have been reported, for which the relationship to troglitazone is unknown, include palpitations, sensations of hot and cold, swelling of body parts, skin eruption, stroke, and hyperglycemia.

Consequently there is a long felt need for a new and combined medicament for the treatment of diabetes, and pre-diabetic, metabolic syndrome, that has fewer, or no, adverse effects (i.e., less toxicity) and favorable profile in terms of blood glucose and lipids.

### SUMMARY OF THE INVENTION

In accordance with one preferred embodiment of the present invention, an oral formulation is disclosed, comprising a therapeutically effective amount of cicletanine in combination with a second agent that lowers blood glucose.

In one preferred variation, the cicletanine comprises a racemic mixture of a (−) and a (+) enantiomers of cicletanine. Alternatively, the cicletanine may be a (−) enantiomer. Alternatively, the cicletanine may be a (+) enantiomer.

In one mode, the second agent is selected from the group consisting of sulfonureas, biguanines, alpha-glucosidase inhibitors, triazolidinediones and meglitinides. Where the second agent is a sulfonurea, it is preferably selected from the group consisting of glimel, glibenclamide; chlorpropamide, tolbutamide, melizide, glipizide and gliclazide. Where the second agent is a biguanine, it is preferably selected from the group consisting of metformin and diaformin. Where the second agent is an alpha-glucosidase inhibitor, it may be selected from the group consisting of: voglibose; acarbose and miglitol. Where the second agent is a thiazolidinedione, it is preferably selected from the group consisting of: pioglitazone, rosiglitazone and troglitazone. Where the second agent is a meglitinide, it may be selected from the group consisting of repaglinide and nateglinide.

In accordance with another embodiment of the present invention, an oral formulation is disclosed, comprising a therapeutically effective amount of cicletanine in combination with a second agent that lowers blood cholesterol.

Preferably, the second agent is selected from the group consisting of: cholestyramine, colestipol, lovastatin, pravastatin, simvastatin, gemfibrozil, clofibrate, nicotinic acid and probucol.

A method for treating and/or preventing complications of diabetes or metabolic syndrome in a mammal is also disclosed. The method comprises administering an oral formulation comprising a therapeutically effective amount of cicletanine and a blood glucose lowering amount of a second agent. Preferably, the second agent is selected from the group consisting of sulfonureas, biguanines, alpha-glucosidase inhibitors, triazolidinediones and meglitinides.

The method is adapted to treat and/or prevent complications selected from the group consisting of retinopathy, neuropathy, nephropathy, microalbuminuria, claudication, macular degeneration, and erectile dysfunction.

In one preferred variation of the method, the therapeutically effective amount of cicletanine is sufficient to mitigate a side effect of said second agent. In another variation, the therapeutically effective amount of cicletanine is sufficient to enhance tissue sensitivity to insulin. Alternatively, the therapeutically effective amount of cicletanine and the blood glucose lowering amount of the second agent are preferably sufficient to produce a synergistic glucose lowering effect.

In another embodiment, a method is disclosed for treating and/or preventing a condition associated with elevated cholesterol in a mammal. The method comprises administering an oral formulation comprising a therapeutically effective amount of cicletanine and a lipid lowering amount of a second agent.

Preferably, the second agent is selected from the group consisting of: cholestyramine, colestipol, lovastatin, pravastatin, simvastatin, gemfibrozil, clofibrate, nicotinic acid and probucol. Alternatively, the second agent is an HMG-CoA reductase inhibitor.

The condition associated with elevated cholesterol is preferably selected from the group consisting of atherosclerosis, hypertension, retinopathy, neuropathy, nephropathy, microalbuminuria, claudication, macular degeneration, and erectile dysfunction.

In accordance with another preferred embodiment of the present invention, a method is disclosed for treating and/or preventing diabetes or metabolic syndrome, comprising administering to a patient in need thereof a therapeutically effective amount of cicletanine, wherein the therapeutically effective amount is sufficient to exert at least two actions selected from the group consisting of lowering blood pressure, decreasing platelet aggregation, lowering blood glucose, lowering total blood cholesterol, lowering LDL cholesterol, lowering blood triglycerides, raising HDL cholesterol, PKC inhibition, and reducing vascular complications associated with diabetes and/or metabolic syndrome.

### DETAILED DESCRIPTION OF THE PREFERRED EMBODIMENT

In an embodiment of the present invention, a combination therapy is disclosed for treating diabetes and metabolic syndrome. The preferred therapy comprises a prostacyclin, an agonist thereof, or an inducer thereof, most preferably cicletanine, in combination with an Oral Antidiabetic Drug selected from sulfonureas, biguanines, alpha-glucosidase inhibitors, triazolidinediones and meglitinides (see Table 1).

TABLE 1
Oral antidiabetic drugs (OAD)

| | Mechanism of | Preferred patient |
|---|---|---|
| Compound (medication) | action | type |
| Sulfonylureas | increase Insulin | Insulinopenic, |
| (Daonil ®, Glimel, Euglocon ® = glibenclamide or | secretion | lean |
| Glyburide ®; Diabinese = Chlorpropamide; | chronically | |
| Rastinon ® = Tolbutamide; Melizide, Glucotrol ®, | | |
| Minidiab ® = glipizide; Diamicron ® = gliclazide) | | |
| Meglitinides | increase Insulin | Hyperglycemic |
| (Repaglinide = Prandin ®, Nateglinide = Starlix ™) | secretion acutely | postprandially |
| α-glucosidase inhibitors (Voglibose; Acarbose = | decrease | Hyperglycemic |
| Glucobay ®; miglitol) | postprandial | postprandially |
| | carbohydrate | |
| | absorption | |
| Biguanidines (Metformin = Glucophage ®; | decrease hepatic | Overweight, with |
| Diabex ®; Diaformin) | glucose | fasting |
| | production | hyperglycemia |
| | decrease insulin | |
| | resistance | |
| Thiazolidinediones, glitazones | decrease insulin | Insulin-resistant, |
| (Actos ® = pioglitazone; Avandia ® = rosiglitazone, | resistance | overweight, |
| Rezulin ® = troglitazone) | decrease hepatic | dyslipidemic and |
| | glucose | renally impaired |
| | production | |
| Insulin | decrease hepatic | Patients with a |
| | glucose | diabetic |
| | production | emergency newly |
| | increase cellular | diagnosed with |
| | uptake of glucose | significant |
| | | hyperglycemia, or |
| | | those with |
| | | hyperglycemia |
| | | despite maximal |
| | | doses of oral |
| | | agents |

Existing oral antidiabetic medicaments to be used in such treatment include the classic insulinotropic agents sulphonylureas (Lebovitz H. E. 1997 “The oral hypoglycemic agents”. In: Ellenberg and Rifkin's Diabetes Mellitus. D. J. Porte and R. S. Sherwin, Editors: Appleton and Lange, p. 761-788). They act primarily by stimulating the sulphonylurea-receptor on the insulin producing beta-cells via closure of the K⁺ATP-sensitive channels.

Alpha-glucosidase inhibitors, such as a carboys, have also been shown to be effective in reducing the postprandial rise in blood glucose (Lefevre, et al. 1992 Drugs 44:29-38). Another treatment used primarily in obese diabetics is metformin, a biguanide.

Compounds useful in the combination therapy discussed above, and methods of making the compounds, are known and some of these are disclosed in U.S. Pat. Nos. 5,223,522 issued Jun. 29, 1993; 5,132,317 issued Jul. 12, 1992; 5,120,754 issued Jun. 9, 1992; 5,061,717 issued Oct. 29, 1991; 4,897,405 issued Jan. 30, 1990; 4,873,255 issued Oct. 10, 1989; 4,687,777 issued Aug. 18, 1987; 4,572,912 issued Feb. 25, 1986; 4,287,200 issued Sep. 1, 1981; 5,002,953, issued Mar. 26, 1991; U.S. Pat. Nos. 4,340,605; 4,438,141; 4,444,779; 4,461,902; 4,703,052; 4,725,610; 4,897,393; 4,918,091; 4,948,900; 5,194,443; 5,232,925; and 5,260,445; WO 91/07107; WO 92/02520; WO 94/01433; WO 89/08651; and JP Kokai 69383/92. The compounds disclosed in these issued patents and applications are useful as therapeutic agents for the treatment of diabetes, hyperglycemia, hypercholesterolemia, and hyperlipidemia. The teachings of these issued patents are incorporated herein by reference in their entireties.

In another embodiment of the present invention, a combination therapy is disclosed for treating diabetes and metabolic syndrome comprising combining a prostacyclin, an agonist thereof, or an inducer thereof, most preferably cicletanine, in combination with a Blood Lipid-Lowering Agent (see Table 2).

TABLE 2
Blood Lipid-Lowering Agents

| Type | Compound/name |
|---|---|
| Resins | Cholestyramine (Cholybar ®, Questran ®); |
| | colestipol (Colestid ®) |
| HMG CoA Reductase | lovastatin (Mevacor ®); |
| Inhibitors | pravastatin (Pravochol ®); |
| | simvastatin (Zocor ®) |
| Fibric Acid Derivatives | gemfibrozil (Lobid); clofibrate |
| | (Atromid-S ®) |
| Miscellaneous | nicotinic acid (Niacin); probucol (Lorelco) |

In another embodiment of the present invention, a combination therapy is disclosed for treating hypertension, and more particularly, for treating and/or preventing the clinical consequences of hypertension, such as nephropathies in hypertensive diabetic patients. The preferred therapy comprises a prostacyclin, an agonist thereof, or an inducer thereof, most preferably cicletanine, in combination with a second antihypertensive agent, selected from the group consisting of diuretics, potassium-sparing diuretics, beta blockers, ACE inhibitors or angiotensin II receptor antagonists, calcium antagonists (preferably second generation, long-acting calcium channel blockers, such as amlodipine), nitric oxide (NO) inducers, and aldosterone antagonists (see Table 3).

TABLE 3
Antihypertensive drugs
Diuretic combinations

| | |
|---|---|
| Amiloride and hydrochlorothiazide (5 mg/50 mg) = | Moduretic ® |
| Spironolactone and hydrochlorothiazide (25 mg/50 mg, 50 mg/50 mg) = | Aldactazide ® |
| Triamterene and hydrochlorothiazide (37.5 mg/25 mg, 50 mg/25 mg) = | Dyazide ® |
| Triamterene and hydrochlorothiazide (37.5 mg/25 mg, 75 mg/50 mg) = | Maxzide-25 mg, |
| | Maxzide ® |

Beta blockers and diuretics

| | |
|---|---|
| Atenolol and chlorthalidone (50 mg/25 mg, 100 mg/25 mg) = | Tenoretic ® |
| Bisoprolol and hydrochlorothiazide (2.5 mg/6.25 mg, 5 mg/6.25 mg, | Ziac ® |
| 10 mg/6.5 mg) = | |
| Metoprolol and hydrochlorothiazide (50 mg/25 mg, 100 mg/25 mg, | Lopressor HCT ® |
| 100 mg/50 mg) = | |
| Nadolol and bendroflumethazide (40 mg/5 mg, 80 mg/5 mg) = | Corzide ® |
| Propranolol and hydrochlorothiazide (40 mg/25 mg, 80 mg/25 mg) = | Inderide ® |
| Propranolol ER and hydrochlorothiazide (80 mg/50 mg, 120 mg/ | Inderide LA ® |
| 50 mg, 160 mg/50 mg) = | |
| Timolol and hydrochlorothiazide (10 mg/25 mg) | Timolide ® |

ACE inhibitors and diuretics

| | |
|---|---|
| Benazepril and hydrochlorothiazide (5 mg/6.25 mg, 10 mg/12.5 mg, | Lotensin HCT ® |
| 20 mg/12.5 mg, 20 mg/25 mg) = | |
| Captopril and hydrochlorothiazide (25 mg/15 mg, 25 mg/25 mg, | Capozide ® |
| 50 mg/15 mg, 50 mg/25 mg) = | |
| Enalapril and hydrochlorothiazide (5 mg/12.5 mg, 10 mg/25 mg) = | Vaseretic ® |
| Lisinopril and hydrochlorothiazide (10 mg/12.5 mg, 20 mg/12.5 mg, | Prinzide ® |
| 20 mg/25 mg) = | |
| Lisinopril and hydrochlorothiazide (10 mg/12.5 mg, 20 mg/12.5 mg, | Zestoretic ® |
| 20 mg/25 mg) = | |
| Moexipril and hydrochlorothiazide (7.5 mg/12.5 mg, 15 mg/25 mg) = | Uniretic ® |

Angiotensin-II receptor antagonists and diuretics

| | |
|---|---|
| Losartan and hydrochlorothiazide (50 mg/12.5 mg, 100 mg/25 mg) = | Hyzaar ® |
| Valsartan and hydrochlorothiazide (80 mg/12.5 mg, 160 mg/12.5 mg) = | Diovan HCT ® |

Calcium channel blockers and ACE inhibitors

| | |
|---|---|
| Amlodipine and benazepril (2.5 mg/10 mg, 5 mg/10 mg, 5 mg/20 mg) = | Lotrel ® |
| Diltiazem and enalapril (180 mg/5 mg) = | Teczem ® |
| Felodipine and enalapril (5 mg/5 mg) = | Lexxel ® |
| Verapamil and trandolapril (180 mg/2 mg, 240 mg/1 mg, 240 mg/ | Tarka ® |
| 2 mg, 240 mg/4 mg) = | |

Miscellaneous combinations

| | |
|---|---|
| Clonidine and chlorthalidone (0.1 mg/15 mg, 0.2 mg/15 mg, 0.3 mg/ | Combipres ® |
| 15 mg) = | |
| Hydralazine and hydrochlorothiazide (25 mg/25 mg, 50 mg/50 mg, | Apresazide ® |
| 100 mg/50 mg) = | |
| Methyldopa and hydrochlorothiazide (250 mg/15 mg, 250 mg/25 mg, | Aldoril ® |
| 500 mg/30 mg, 500 mg/50 mg) = | |
| Prazosin and polythiazide (1 mg/0.5 mg, 2 mg/0.5 mg, 5 mg/0.5 mg) = | Minizide ® |

The combination may be formulated in accordance with the teachings herein to provide a clinical benefit that goes beyond the beneficial effects produced by either drug alone. Such an enhanced clinical benefit may be related to distinct mechanisms of action and/or a synergistic interaction of the drugs.

In one preferred embodiment, the combination therapy includes in addition to the prostacyclin, a phosphodiesterase (PDE) inhibitor, which stabilizes cAMP (second messenger for prostacyclins), and may amplify the vasodilatory and/or nephroprotective actions of the prostacyclin agonist or inducer. In another preferred embodiment, the combination therapy comprises cicletanine and amlodipine. In another preferred embodiment, the combination therapy comprises cicletanine and an ACE inhibitor or angiotensin II receptor antagonist. In another preferred embodiment, the combination therapy comprises cicletanine and a thiazolidinedione (e.g., rosiglitazone, pioglitazone), which is known to be a ligand of the peroxisome proliferator-activated receptor gamma (PPARgamma). In another embodiment, the combination therapy comprises cicletanine and a peroxisome proliferator-activated receptor (PPAR) agonist, including but not limited to agonists of one or more of the following types: alpha, gamma and delta). In another embodiment, the combination therapy comprises cicletanine and a sulfonurea (e.g., glibenclamide, tolbutamide, melizide, glipiziede, gliclazide). In another preferred embodiment, the combination therapy comprises cicletanine and a meglitinide (e.g., repaglinide, nateglinide). In another preferred embodiment, the combination therapy comprises cicletanine and a biguanide (e.g., metformin, diaformin). In another preferred embodiment, the combination therapy comprises cicletanine and a lipid-lowering agent.

The combination therapy preferably comprises a fixed dose (of each component), oral dosage formulation (e.g., single tablet, capsule, etc.), which provides a systemic action (e.g., blood pressure-lowering, organ-protective, glucose-lowering, lipid-lowering, etc.), with minimal side effects. The rationale for using a fixed-dose combination therapy in accordance with a preferred embodiment of the present invention is to obtain sufficient blood pressure control by employing an antihypertensive agent, e.g., cicletanine, which also lowers blood glucose and LDLs, while enhancing compliance by using a single tablet that is taken once or twice daily. Using low doses of different agents can also minimize the clinical and metabolic effects that occur with maximal dosages of the individual components of the combined tablet.

In addition to the advantages resulting from two distinct mechanisms of action, some drug combinations produce potentially synergistic effects. For example, Vaali K. et al. 1998 (Eur J Pharmacol 363:169-174) reported that the β2 agonist, salbutamol, in combination with micromolar concentrations of NO donors, SNP and SIN-1, caused a synergistic relaxation in metacholine-induced contraction of guinea pig tracheal smooth muscle.

In one aspect, the combination may be formulated to generate an enhanced clinical benefit which is related to the diminished side-effect(s) of one or both of the drugs. For example, one significant side-effect of calcium antagonists, such as amlodipine (Norvasc R®), the most commonly prescribed calcium channel blocker, is edema in the legs and ankles. In contrast, cicletanine has been shown to cause significant and major improvement in edema of the lower limbs (Tarrade et al. 1989 Arch Mal Couer Vaiss 82 Spec No. 4:91-7). Thus, in addition to their distinct antihypertensive actions the combination of cicletanine and amlodipine may be particularly beneficial as a result of diminished edema in the lower limbs. In another example, aldosterone antagonists may cause hyperkalemia and cicletanine in high doses causes potassium excretion. Thus, the combination of cicletanine and an aldosterone antagonist may relieve hyperkalemia, a potential side effect of the aldosterone inhibitor alone. In yet another example, thiazolidinediones (aka glitazones), of which there are two marketed in the US: Rosiglitazone (Avandia®) and Pioglitazone (Actos®), are effective in lowering blood glucose), but they have diverging effects on LDL. Actos® tends to reduce LDL, while Avandia® tends to increase LDL (Viberti G. C. 2003 Int J Clin Pract 57:128-34; Ko S. H. et al. 2003 Metabolism 52:731-4; Raji A. et al. 2003 Diabetes Care 26:172-8). Thiazolidinediones also known to cause weight gain and fluid retention. The combination of cicletanine with thiazolidinediones is envisioned to control the lipid metabolism and the fluid retention, due to the differences in the mechanism of action of the named compounds. Moreover, the thiazolidinediones tend to be hepatotoxic. The composition of the present invention will allow to lower the thiazolidinediones dose necessary to achieve a comparable level of insulin sensitization and glucose control, thereby reducing the risk of hepatotoxicity.

### Prostacyclins

In a broad sense, the prostacyclin included as a first agent in a preferred embodiment of the combination therapy can be selected from the group consisting of any eicosanoids, including agonists, analogs, derivatives, mimetics, or inducers thereof, which exhibit vasodilatory effects. Some eicosanoids, however, such as the thromboxanes have opposing vasoconstrictive effects, and would therefore not be preferred for use in the inventive formulations. The eicosanoids are defined herein as a class of oxygenated, endogenous, unsaturated fatty acids derived from arachidonic acid. The eicosanoids include prostanoids (which refers collectively to a group of compounds including the prostaglandins, prostacyclins and thromboxanes), leukotrienes and hydroxyeicosatetraenoic acid compounds. They are hormone-like substances that act near the site of synthesis without altering functions throughout the body.

The prostanoids (prostaglandins, prostacyclins and thromboxanes) are any of a group of components derived from unsaturated 20-carbon fatty acids, primarily arachidonic acid, via the cyclooxygenase (COX) pathway that are extremely potent mediators of a diverse group of physiologic processes. The prostaglandins (PGs) are designated by adding one of the letters A through I to indicate the type of substituents found on the hydrocarbon skeleton and a subscript (1, 2 or 3) to indicate the number of double bonds in the hydrocarbon skeleton for example, PGE₂. The predominant naturally occurring prostaglandins all have two double bonds and are synthesized from arachidonic acid (5, 8, 11, 14 eicosatetraenoic acid). The 1 series and 3 series are produced by the same pathway with fatty acids having one fewer double bond (8, 11, 14 eicosatrienoic acid or one more double bond (5, 8, 11, 14, 17 eicosapentaenoic acid) than arachidonic acid. The prostaglandins act by binding to specific cell surface receptors causing an increase in the level of the intracellular second messenger cyclic AMP (and in some cases cyclic GMP). The effect produced by the cyclic AMP increase depends on the specific cell type. In some cases there is also a positive feedback effect. Increased cyclic AMP increases prostaglandin synthesis leading to further increases in cyclic AMP.

Prostaglandins have a variety of roles in regulating cellular activities, especially in the inflammatory response where they may act as vasodilators in the vascular system, cause vasoconstriction or vasodilatation together with bronchodilation in the lung and act as hyperalgesics. Prostaglandins are rapidly degraded in the lungs and will not therefore persist in the circulation.

Prostacyclin, also known as PGI₂, is an unstable vinyl ether formed from the prostaglandin endoperoxide, PGH₂. The conversion of PGH₂ to prostacyclin is catalyzed by prostacyclin synthetase. The two primary sites of synthesis are the veins and arteries. Prostacyclin is primarily produced in vascular endothelium and plays an important inhibitory role in the local control of vascular tone and platelet aggregation. Prostacyclin has biological properties opposing the effect of thromboxane A₂. Prostacyclin is a vasodilator and a potent inhibitor of platelet aggregation whereas thromboxane A₂ is a vasoconstrictor and a promoter of platelet aggregation. A physiological balance between the activities of these two effectors is probably important in maintaining a healthy blood supply.

In one aspect of the present combination therapy, the relative dosages and administration frequency of the prostacyclin agent and the second therapeutic agent may be optimized by monitoring the thromboxane/PGI₂ ratio. Indeed, it has been observed that this ratio is significantly increased in diabetics compared to normal individuals, and even higher in diabetic with retinopathy (Hishinuma et al. 2001 Prostaglandins, Leukotrienes and Essential Fatty Acids 65(4): 191-196). The thromboxane/PGI₂ ratio may be determined as detailed by Hishinuma et al., (2001) by measuring the levels (pg/mg) in urine of 11-dehydro-thromboxane B₂ and 2,3-dinor-6-keto-prostaglandin F_{1α}, the urinary metabolites of thromboxane A₂ and prostacyclin, respectively. Hishinuma et al. found that the thromboxane/PGI₂ ratio in healthy individuals was 18.4±14.3. In contrast, the thromboxane/PGI₂ ratio in diabetics was 52.2±44.7. Further, the thromboxane/PGI₂ ratio was even higher in diabetics exhibiting microvascular complications, such as retinopathy (75.0±67.8). Accordingly, optimization of relative dosages and administration frequencies would target thromboxane/PGI₂ ratios of less than about 50, and more preferably between about 20 and 50, and most preferably, about 20. Of course, the treating physician would also monitor a variety of indices, including blood glucose, blood pressure, lipid profiles, impaired clotting and/or excess bleeding, as well known by those of skill in the art.

Prostacyclin Agonists—

Prostacyclin is unstable and undergoes a spontaneous hydrolysis to 6-keto-prostaglandin F1α (6-keto-PGF1α). Study of this reaction in vitro established that prostacyclin has a half-life of about 3 min. Because of its low stability, several prostacyclin analogues have been synthesized and studied as potential therapeutic compounds. One of the most potent prostacyclin agonists is iloprost, a structurally related synthetic analogue of PGI₂. Cicaprost is closely related to iloprost and possess a higher degree of tissue selectivity. Both iloprost and cicaprost are amenable to oral delivery and provide extended half-life. Other prostacyclin analogs include beraprost, epoprostenol (Flolan®) and treprostinil (Remodulin®).

Prostacyclin plays an important role in inflammatory glomerular disorders by regulating the metabolism of glomerular extracellular matrix (Kitahara M. et al. 2001 Kidney Blood Press Res 24:18-26). Cicaprost attenuated the progression of diabetic renal injury, as estimated by lower urinary albumin excretion, renal and glomerular hypertrophies, and a better renal architectural preservation. Cicaprost also induced a significant elevation in renal plasma flow and a significant decrease in filtration fraction. These findings suggest that oral stable prostacyclin analogs could have a protective renal effect, at least in this experimental model (Villa E. et al. 1993 Am J Hypertens 6:253-7).

In a follow-up study, Villa et al. (Am J Hypertens 1997 10:202-8), found that chronic therapy with cicaprost, fosinopril (an ACE inhibitor), and the combination of both drugs, stopped the progression of diabetic renal injury in an experimental rat model of diabetic nephropathy (uninephrectomized streptozotocin-induced diabetic rats). Control rats exhibited characteristic features of this model, such as high blood pressure and plasma creatinine and urinary albumin excretion, together with prominent alterations in the kidney (renal and glomerular hypertrophies, mesangial matrix expansion, and tubular alterations). The three therapies attenuated equivalently the progression of diabetic renal injury, as estimated by lower urinary albumin excretion, renal and glomerular hypertrophies, and a better renal architectural preservation. No synergistic action was observed with the combined therapy. However, renal preservation achieved with cicaprost was not linked to reductions in systemic blood pressure, whereas in the groups treated with fosinopril the hypotensive effect of this drug could have contributed to the positive outcome of the therapy. The authors speculated that impaired prostacyclin synthesis or bioavailability may have been involved in the pathogenesis of the diabetic nephropathy in this model.

Cicletanine—

Cicletanine is a drug that increases endogenous prostacyclin levels. It was originally developed as an antihypertensive agent that has diuretic properties at high doses. Cicletanine is produced as two enantiomers [(−)- and (+)-cicletanine] which independently contribute to the vasorelaxant and natriuretic mechanisms of this drug. The renal component of the antihypertensive action of cicletanine appears to be mediated by (+)-cicletanine sulfate. It has been shown in animal models and in vitro that the (−)enantiomer is primarily responsible for vasorelaxant activity and has more potent cardioprotective activity.

1) (−) contributes to antihypertensive activity by reducing the vascular reactivity to endogenous pressor substances such as angiotensin II and vasopressin (Alvarez-Guerra et al. 1996 J Cardvascular Pharmacol 28:564-70).

2) (−)-enantiomer reduced the Et-1 (endothelin-1) dependent vasoconstriction more potently that (+)-cicletanine. This observation in the human artery is in agreement with the earlier animal in vivo and in vitro data demonstrating greater vasorelaxant properties of (−)-cicletanine versus action of the (+)-enantiomer (Bagrov A. Y. et al., 1998 Am J Hypertens 11 (11 Pt 1):1386-9).

3) Both enantiomers had cardioprotective effects. The (−) enantiomer had greater protective effect (anti-ischemic and antiarrythmic). The antiarrythmic action of (−) cicletanine may be of particular significance in combination therapies involving sulfonylureas, some of which have been associated with an increased incidence of cardiac arrhythmias.

Cicletanine is a furopyridine antihypertensive drug which exhibits three major effects, vasorelaxation, natriuretic and diuretic, and organ protection (Kalinowski L. et al. 1999 Gen Pharmacol 33:7-16). One of the attractive properties of cicletanine is its safety and absence of serious side effects (Tarrade T. & Guinot P. 1988 Drugs Exp Clin Res 14:205-14). Cicletanine has several mechanisms of action. Its natriuretic activity is attributed to inhibition of apical Na⁺-dependent Cl⁻/HCO₃⁻ anion exchanger in the distal convoluted tubule (Garay R. P. et al. 1995 Eur J Pharmacol 274:175-80). The nature of vasorelaxant activity of cicletanine is more complex and involves inhibition of low Kₘ cGMP phosphodiesterases (Silver P. J. et al. 1991 J Pharmacol Exp Ther 257:382-91), stimulation of vascular NO synthesis (Hirawa N. et al. 1996 Hypertens Res 19:263-70), inhibition of PKC (Silver P. J. et al. 1991 J Pharmacol Exp Ther 257:382-91; Bagrov A. Y. et al. 2000 J Hypertens 8:209-15), and antioxidant activity (Uehara Y. et al. 1993 Am J Hypertens 6 (6 Pt 1):463-72). Combination of the above effects explains the results of numerous clinical and experimental reports regarding the most promising feature of cicletanine, i.e., organ protection (renal, vascular, and ocular).

Natriuretic and Diuretic Activity—

In healthy subjects and nonhypertensive experimental animals cicletanine exhibits moderate diuretic and natriuretic effects (Kalinowski L. et al. 1999 Gen Pharmacol 33:7-16; Moulin B. et al. 1995 J Cardiovasc Pharmacol 25:292-9). In the hypertensives, however, cicletanine does induce natriuresis without affecting plasma potassium levels, although its effect is milder than that of thiazide diuretics (Singer D. R. et al. 1990 Eur J Clin Pharmacol 39:227-32). However, to it is unclear to what extent natriuretic properties of cicletanine in the hypertensives are related to its renoprotective (vs. direct renotubular) effect.

In the late 1980's several clinical studies were aimed towards assessment of antihypertensive efficacy of cicletanine. In a multicenter trial 1050 hypertensives were administered 50 mg/kg cicletanine for three months (Tarrade T. & Guinot P. 1988 Drugs Exp Clin Res 14:205-14). In one third of patients the dose was doubled. The blood pressure decreased from 176/104 to 151/86 (Tarrade T. & Guinot P. 1988 Drugs Exp Clin Res 14:205-14). In another study, in a group of patients whose blood pressure had not been normalized by calcium channel blockers, beta blockers and ACE inhibitors, cicletanine (50 and 100 mg per day) has been tested in combination with the above drugs (Tarrade T. et al. 1989 Arch Mal Coeur Vaiss 82 Spec No 4:103-8). The addition of cicletanine normalized the blood pressure in 50% of patients from all three groups without major adverse effects. In experimental studies, cicletanine also proved effective with respect to lowering the blood pressure (Fuentes J. A. et al. 1989 Am J Hypertens 2:718-20; Ando K. et al. 1994 Am J Hypertens 7:550-4). Remarkably, cicletanine proved especially effective in the models of NaCl sensitive hypertension (Jin H. K. et al. 1991 Am J Med Sci 301:383-9), and its action was associated with antiremodeling effects (Chabrier P. E. et al. 1993 J Cardiovasc Pharmacol 21 Suppl 1:S50-3; Fedorova O. V. et al. 2003 Hypertension 41:505-11).

The most convincing body of evidence arises from the studies demonstrating organ protection induced by cicletanine in various experimental models. In spontaneously hypertensive rats, cicletanine, in the face of comparable blood pressure lowering effect, showed better protection of myocardium and vasculature than captopril (Ruchoux M. M. et al. 1989 Arch Mal Coeur Vaiss 82 Spec No 4:169-74). In NaCl sensitive Dahl rats rendered hypertensive cicletanine treatment produced reduction of blood pressure, medial mass regression of the vascular wall, attenuated glomerular sclerosis and enhanced GFR and natriuresis, restored the endothelial NO production, and produced beneficial metabolic effects including reduction in plasma levels of low-density lipoprotein and a concomitant increase in high-density lipoprotein (Fedorova et al. 2003 Hypertension 41:505-11; Uehara Y. et al. 1997 Blood Press 6:180-7; Uehara Y. et al. 1991 J Hypertens 9:719-28; Uehara Y. et al. 1991 J Cardiovasc Pharmacol 18:158-66). In rats with streptozotocin induced diabetes mellitus the non-depressor dose of cicletanine exhibited renal protective effect on both functional and morphological levels and reduced the heart weight to body weight ratio (Kohzuki M. et al. 1999 J Hypertens 17:695-700; Kohzuki M, et al. 2000 Am J Hypertens 13:298-306).

It is well known that excessive NaCl intake is a risk factor for insulin resistance, and insulin resistance, vice versa, is frequently associated with the development of NaCl sensitive hypertension (Galletti F. et al. 1997 J Hypertens 15:1485-1492; Ogihara T. et al. 2003 Life Sci 73: 509-523). The exaggerated efficacy of cicletanine in sodium dependent hypertension, as well as the ability of cicletanine to improve kidney function in experimental diabetes mellitus, make this drug potentially very attractive for treatment of hypertension in diabetics, patients with metabolic and cardiac syndrome X, and hypertensives with impaired glucose tolerance.

Many molecular mechanisms underlie hypertrophic signaling in the cardiovascular system in diabetics, including PKC signaling (Nakamura J. et al. 1999 Diabetes 48:2090-5; Meier M. & King G. L. 2000 Vasc Med 5:173-85) and dysregulation of the Na/K-ATPase (Ottlecz A. et al. 1996 Invest Ophthalmol Vis Sci 37:2157-64; Chan J. C. et al. 1998 Lancet 351:266), which, in turn, initiates several cascades of growth promoting signaling (Kometiani P. et al. 1998 J Biol Chem 273:15249-15267). Moreover, inhibition of beta-2 isoform of the PKC is thought to be a promising direction in the treatment of diabetic complications (Meier M. & King G. L. 2000 Vasc Med 5:173-85). Recently, cicletanine has been reported to inhibit PKC (Bagrov A. Y. et al. 2000 J Hypertens 8:209-15) and to restore the Na/K-ATPase in hypertensive Dahl rats (Fedorova O. V. et al. 2003 Hypertension 41:505-11). Remarkably, treatment of these Dahl-S rats with 30 mg/kg/day cicletanine prevented the upregulation of beta-2 PKC in the myocardial sarcolemma.

Although cicletanine has never been specifically studied in the diabetics, data from earlier clinical studies provide information which indicates that cicletanine exhibits beneficial metabolic effects. In 1988 in a multicenter clinical trial three-month administration of cicletanine resulted in the lowering of plasma glucose, cholesterol, and triglycerides (Tarrade T. & Guinot P. 1988 Drugs Exp Clin Res 14:205-14). Similar results were obtained from a study of a higher dose of cicletanine (mean daily dose of 181 mg) in 52 hypertensive patients.

A very intriguing observation has been made by Bayes et al., who studied interaction between cicletanine and a hypoglycemic drug, tolbutamide (Bayes M. C. et al. 1996 Eur J Clin Pharmacol 50:381-4). In this study, in 10 healthy subjects, an effect of a single intravenous dose of tolbutamide on plasma levels of glucose and insulin has been studied alone and following 7 days of administration of cicletanine (100 mg per day). Administration of tolbutamide was associated with a decrease in blood glucose levels and with a parallel rise in plasma immunoreactive insulin. Remarkably, following cicletanine administration, the hypoglycemic effect of tolbutamide did not change, although peak insulin response was much less than before cicletanine administration (17.4 and 29.2 mU/L, respectively). Thus, in the presence of cicletanine tissue insulin sensitivity has been increased. The ability to improve the insulin sensitivity appears to be consistent with the ability of cicletanine to inhibit PKC, which is involved in the mechanisms of tissue insulin resistance (Kawai Y. et al. 2002 IUBMB Life 54:365-70; Abiko T. et al. 2003 Diabetes 52:829-37; Schmitz-Peiffer C. 2002 Ann N Y Acad Sci 967:146-57).

The above indicates that cicletanine, due to a unique combination of several properties: vasorelaxation, natriuresis, renal protection, improvement of endothelial function, inhibition of PKC, improvement of glucose/insulin metabolism, may be especially effective as a monotherapy and in combination with the other drugs in the hypertensive patients with diabetes mellitus and metabolic syndrome.

The efficacy of a combination of cicletanine (100 mg per day) with a second agent such as an antihypertensive agent (an ACE inhibitor, angiotensin II receptor antagonist, beta blocker, calcium channel blocker, etc.), or an Oral Antidiabetic (a sulfonurea, biguanines, an alpha-glucosidase inhibitor, a triazolidinedione or a meglitinide), or a lipid-lowering agent (a resin, an HMG CoA Reductase Inhibitor, a Fibric Acid Derivative, or nicotinic acid, or probucol) can be assessed in a pilot study in the hypertensives with and without type 1 or 2 diabetes mellitus or metabolic syndrome. The major endpoints of such a study would be effects of blood pressure, left ventricular function, insulin sensitivity, blood glucose, HDL levels, LDL levels, and renal functions.

Cicletanine (39 mg/kg body weight per day for 6 weeks) ameliorated the development of hypertension in Dahl-S rats fed a high-salt (4% NaCl) diet. This blood pressure reduction was associated with a decrease in heart weight and vascular wall thickness. Moreover, urinary prostacyclin (PGI₂) excretion was increased with cicletanine treatment, being inversely related to systolic blood pressure. Proteinuria and urinary excretion of n-acetyl-beta-D-glucosaminidase were decreased and glomerular filtration rate was increased with this treatment. Morphological investigation revealed an improvement in glomerulosclerosis, renal tubular damage and intrarenal arterial injury in the salt-induced hypertensive rats. Thus, these data indicate that cicletanine ameliorates the development of hypertension in Dahl-S rats and protects the cardiovascular and renal systems against the injuries seen in the hypertension (Uehara Y, et al. 1991 J Hypertens 9:719-28).

In another study, cicletanine-treated rats exhibited a 56-mm Hg reduction in blood pressure (P<0.01) and a 30% reduction in left ventricular weight, whereas cardiac alpha-1 Na/K-ATPase protein and (Marinobufagenin) MBG levels were unchanged. In cicletanine-treated rats, protein kinase C(PKC) beta2 was not increased, the sensitivity of Na/K-ATPase to MBG was decreased (IC₅₀=20 micromol/L), and phorbol diacetate-induced alpha-1 Na/K-ATPase phosphorylation was reduced versus vehicle-treated rats. In vitro, cicletanine treatment of sarcolemma from vehicle-treated rats also desensitized Na/K-ATPase to MBG, indicating that this effect was not solely attributable to a reduction in blood pressure. Thus, PKC-induced phosphorylation of cardiac alpha-1 Na/K-ATPase is a likely target for cicletanine action (Fedorova O. V et al. 2003 Hypertension 41:505-11).

In another set of studies, Kohzuki et al. (Am J Hypertens 2000 13:298-306; and J Hypertens 1999 17:695-700) assessed the renal and cardiac benefits of cicletanine in different rat models exhibiting diabetic hypertension with renal impairment. The authors reported that cicletanine treatment significantly and effectively protected against an increase in the index of focal glomerular sclerosis in the diabetic rat models. Moreover, cicletanine treatment significantly attenuated the increase in the heart weight to body weight ratio in these diabetic rats. Treatment with cicletanine did not affect urinary and blood glucose concentrations at the protective dosage. These results suggest that cicletanine has a renal-protective action, which is not related to improvement of diabetes or improvement of high blood pressure in diabetic rats with hypertension.

### Nephroprotective Mechanisms of Action of Prostacyclins

Although the renal protective mechanism of action of prostacyclins and prostacyclin inducers is largely unknown, there are at present numerous theories. For example, Kikkawa et al. (Am J Kidney Dis 2003 41 (3 Suppl 2):S19-21), have postulated that the PKC-MAPK pathway may play an important role in prostacyclin-mediated nephroprotection. They examined whether inhibition of the PKC-MAPK pathway could inhibit functional and pathological abnormalities in glomeruli from diabetic animal models and cultured mesangial cells exposed to high glucose condition and/or mechanical stretch. The authors reported that direct inhibition of PKC by PKC beta inhibitor prevented albuminuria and mesangial expansion in db/db mice, a model of type 2 diabetes. They also found that inhibition of MAPK by PD98059, an inhibitor of MAPK, or mitogen-activated extracellular regulated protein kinase prevented enhancement of activated protein-1 (AP-1) DNA binding activity and fibronectin expression in cultured mesangial cells exposed to mechanical stretch in an in vivo model of glomerular hypertension. These findings highlight the potential role of PKC-MAPK pathway activation in mediating the development and progression of diabetic nephropathy.

There is compelling evidence for endothelial dysfunction in both type 1 and type 2 diabetics (See e.g., Taylor, A. A. 2001 Endocrinol Metab Clin North Am 30:983-97). This dysfunction is manifest as blunting of the biologic effect of a potent endothelium-derived vasodilator, nitric oxide (NO), and increased production of vasoconstrictors such as angiotensin II, ET-1, and cyclooxygenase and lipoxygenase products of arachidonic acid metabolism. These agents and other cytokines and growth factors whose production they stimulate cause acute increases in vascular tone, resulting in increases in blood pressure, and vascular and cardiac remodeling that contributes to the microvascular, macrovascular, and renal complications in diabetes. Reactive oxygen species, overproduced in diabetics, may serve as signaling molecules that mediate many of the cellular biochemical reactions that result in these deleterious effects. Adverse vascular consequences associated with endothelial dysfunction in diabetes mellitus include: decreased NO formation, release, and action; increased formation of reactive oxygen species; decreased prostacyclin formation and release; increased formation of vasoconstrictor prostanoids; increased formation and release of ET-1; increased lipid oxidation; increased cytokine and growth factor production; increased adhesion molecule expression; hypertension; changes in heart and vessel wall structure; and acceleration of the atherosclerotic process. Treatment with antioxidants and ACE inhibitors may reverse some of the pathologic vascular changes associated with endothelial dysfunction. Further, since prostacyclins enhance NO release and exert direct vasodilatory effects, treatment with prostacyclin agonists or inducers should be effective in protecting against and possibly reversing vascular changes associated with diabetic glomerulosclerosis.

Based on the study of Villa et al. (Am J Hypertens 1997 10:202-8), Applicants have inferred that cicletanine plus an ACE inhibitor could provide a preferred combination therapy in treating diabetes patients with hypertension. Indeed, cicletanine produced positive results in diabetic animal models alone and in combination with the ACE inhibitor, fosinopril, (See e.g., Villa et al. 1997 Am J Hypertens 10:202-8). Similarly, cicletanine has been shown in unpublished results to reduce microalbuminuria in diabetic humans. Cicletanine is also suggested as a drug of choice in diabetics because it inhibits the beta isoform of PKC, and such inhibition has been demonstrated effective against diabetic complications in animal models, and increasingly, in human clinical trials. Another reason for using cicletanine in combination with an ACE inhibitor is the predicted balance between cicletanine's enhancement of potassium excretion and the mild retention of potassium typically seen with ACE inhibitors.

Another therapeutic approach is the use of PKC inhibitors such as LY333531. Cicletanine is particularly interesting in this regard because of evidence that it has, at least in some populations, a three-fold action of glycemic control, blood-pressure reduction and PKC inhibition. The combination of cicletanine with a commonly-used antihypertensive medication is therefore a promising approach to treating hypertension, particularly in patients with diabetes or metabolic syndrome.

Prostacyclin Delivery and Side Effects—

Clinical experiences with prostacyclin agonists have been significantly documented in treatment of primary pulmonary hypertension (PPH). The lessons learned in treating PPH may be valuable in developing prostacyclin-mediated therapies for treatment and/or prevention of diabetic complications (e.g., nephropathy, retinopathy, neuropathy, etc.). Prostacyclin agonists, such as epoprostenol (Flolan®), have been delivered by injection through a catheter into the patient, usually near the gut. The drug is slowly absorbed after being injected into fat cells. These agonists have been shown to exert direct effects the blood vessels of the lung, relaxing them enabling the patient to breathe easier. This treatment regimen is used for primary pulmonary hypertension. Some researchers believe it may also slow the PPH scarring process. The intravenous prostacyclin agonist, epoprostenol, has been shown to improve survival, exercise capacity, and hemodynamics in patients with severe PPH.

Side effects typically seen in patients receiving prostacyclins (agonists or inducers) include headache, jaw pain, leg pain, and diarrhea, and there may be complications with the injection delivery system. These findings are well documented for continuous intravenous epoprostenol therapy and have also been reported with the subcutaneous delivery of the prostacyclin preparation treprostinil. Oral application of the prostacyclin agonist, beraprost, may decrease delivery-associated risks, but this delivery route has not yet been shown to be effective in severe disease, although in moderately ill PPH patients, there was a significant benefit in a controlled study.

Aerosolization of prostacyclin and its stable analogues caused selective pulmonary vasodilation, increased cardiac output and improved venous and arterial oxygenation in patients with severe pulmonary hypertension. However, the severe vasodilator action of prostacyclin and its analogs also produced severe headache and blood pressure depression. Nevertheless, inhaled prostacyclins have shown promise for the treatment of pulmonary arterial hypertension (Olschewski, et al. 1999 Am J Respir Crit Care Med. 160:600-7). Inhaled prostacyclin therapy for pulmonary hypertension may offer selectivity of hemodynamic effects for the lung vasculature, thus avoiding systemic side effects.

PDE's Potentiate Prostacyclin Activity—

Although aerosolized prostacyclin (PGI₂) has been suggested for selective pulmonary vasodilation as discussed above, its effect rapidly levels off after termination of nebulization. Stabilization of the second-messenger cAMP by phosphodiesterase (PDE) inhibition has been suggested as a strategy for amplification of the vasodilative response to nebulized PGI₂. Lung PDE3/4 inhibition, achieved by intravascular or transbronchial administration of subthreshold doses of specific PDE inhibitors, synergistically amplified the pulmonary vasodilatory response to inhaled PGI₂, concomitant with an improvement in ventilation-perfusion matching and a reduction in lung edema formation. The combination of nebulized PGI₂ and PDE3/4 inhibition may thus offer a new concept for selective pulmonary vasodilation, with maintenance of gas exchange in respiratory failure and pulmonary hypertension (Schermuly R. T. et al. 2000 J Pharmacol Exp Ther 292:512-20).

A phosphodiesterase (PDE) inhibitor is any drug used in the treatment of congestive cardiac failure (CCF) that works by blocking the inactivation of cyclic AMP and acts like sympathetic simulation, increasing cardiac output. There are five major subtypes of phosphodiesterase (PDE); the drugs enoximone (inhibits PDE IV) and milrinone (Primacor®) (inhibits PDE IIIc) are most commonly used medically. Other phosphodiesterase inhibitors include sildenafil (Viagra®); a PDE V inhibitor used to treat neonatal pulmonary hypertension) and Amrinone (Inocor®) used to improve myocardial function, pulmonary and systemic vasodilation.

Isozymes of cyclic-3′,5′-nucleotide phosphodiesterase (PDE) are a critically important component of the cyclic-3′,5′-adenosine monophosphate (cAMP) protein kinase A (PKA) signaling pathway. The superfamily of PDE isozymes consists of at least nine gene families (types): PDE1 to PDE9. Some PDE families are very diverse and consist of several subtypes and numerous PDE isoform-splice variants. PDE isozymes differ in molecular structure, catalytic properties, intracellular regulation and location, and sensitivity to selective inhibitors, as well as differential expression in various cell types. Type 3 phosphodiesterases are responsible for cardiac function

A number of type-specific PDE inhibitors have been developed. Current evidence indicates that PDE isozymes play a role in several pathobiologic processes in kidney cells. Administration of selective PDE isozyme inhibitors in vivo suppresses proteinuria and pathologic changes in experimental anti-Thy-1.1 mesangial proliferative glomerulonephritis in rats. Increased activity of PDE5 (and perhaps also PDE9) in glomeruli and in cells of collecting ducts in sodium-retaining states, such as nephrotic syndrome, accounts for renal resistance to atriopeptin; diminished ability to excrete sodium can be corrected by administration of the selective PDE5 inhibitor zaprinast. Anomalously high PDE4 activity in collecting ducts is a basis of unresponsiveness to vasopressin in mice with hereditary nephrogenic diabetes insipidus. PDE isozymes are a target for action of numerous novel selective PDE inhibitors, which are key components in the design of novel “signal transduction” pharmacotherapies of kidney diseases (Dousa T. P. 1999 Kidney Int 55:29-62).

Nitric Oxide (NO) Donors/Inducers—

NO is an important signaling molecule that acts in many tissues to regulate a diverse range of physiological processes. One role is in blood vessel relaxation and regulating vascular tone. Nitric oxide is a short-lived molecule (with a half-life of a few seconds) produced from enzymes known as nitric oxide synthasases (NOS). Since it is such a small molecule, NO is able to diffuse rapidly across cell membranes and, depending on the conditions, is able to diffuse distances of more than several hundred microns. The biological effects of NO are mediated through the reaction of NO with a number of targets such as heme groups, sulfhydryl groups and iron and zinc clusters. Such a diverse range of potential targets for NO explains the large number of systems that utilize it as a regulatory molecule.

The earliest medical applications of NO relate to the function of NOS in the cardiovascular system. Nitroglycerin was first synthesized by Alfred Nobel in the 1860s, and this compound was eventually used medicinally to treat chest pain. The mechanism by which nitrovasodilators relax blood vessels was not well defined but is now known to involve the NO signaling pathway. Cells that express NOS include vascular endothelial cells, cardiomyocytes and others. In blood vessels, NO produced by the NOS of endothelial cells functions as a vasodilator thereby regulating blood flow and pressure. Mutant NOS knockout mice have blood pressure that is 30% higher than wild-type littermates. Within cardiomyocytes, NOS affects Ca²⁺ currents and contractility. Expression of NOS is usually reported to be constitutive though modest degrees of regulation occur in response to factors such as shear stress, exercise training, chronic hypoxia, and heart failure.

The unique N-terminal sequence of NOS is about 70 residues long and functions to localize the enzyme to membranes. Upon myristoylation at one site and palmitoylation at two other sites within this segment, the enzyme is exclusively membrane-bound. Palmitoylation is a reversible process that is influenced by some agonists and is essential for membrane localization. Within the membrane, NOS is targeted to the caveolae, small invaginations characterized by the presence of proteins called caveolins. These regions serve as sites for the sequestration of signaling molecules such as receptors, G proteins and protein kinases. The oxygenase domain of NOS contains a motif that binds to caveolin-1, and calmodulin is believed to competitively displace caveolin resulting in NOS activation. Bound calmodulin is required for activity of NOS, and this binding occurs in response to transient increases in intracellular Ca²⁺. Thus, NOS occurs at sites of signal transduction and produces short pulses of NO in response to agonists that elicit Ca²⁺ transients. Physiological concentrations of NOS-derived NO are in the picomolar range.

Within the cardiovascular system, NOS generally has protective effects. Studies with NOS knockout mice clearly indicate that NOS plays a protective role in cerebral ischemia by preserving cerebral blood flow. During inflammation and atherosclerosis, low concentrations of NO prevent apoptotic death of endothelial cells and preserve the integrity of the endothelial cell monolayer. Likewise, NO also acts as an inhibitor of platelet aggregation, adhesion molecule expression, and vascular smooth muscle cell proliferation. Therefore, NOS-related pathologies usually result from impaired NO production or signaling. Altered NO production and/or bioavailability have been linked to such diverse disorders as hypertension, hypercholesterolemia, diabetes, and heart failure.

Cicletanine's vasorelaxant and vasoprotective properties may be mediated by its effects on nitric oxide and superoxide. It was been shown in situ that cicletanine stimulates NO release in endothelial cells at therapeutic concentrations. (Kalinowski, et al. 2001 J Vascular Pharmacol 37:713-724). NO release was observed at concentrations similar to the plasma concentrations obtained following dosing with 75-200 mg of cicletanine. While cicletanine stimulates both NO release and release of O₂⁻, cicletanine scavenges superoxide at nanomolar levels. Thus, cicletanine is able to increase the net production of diffusible NO. These effects may contribute to the potent vasorelaxation properties of cicletanine.

Superoxide consumes NO to produce peroxynitrite (OONO⁻) which in turn may undergo cleavage to produce OH, NO₂ radicals and NO₂⁺, which are among the most reactive and damaging species in biological systems. Cicletanine prevents production of these damaging species both by its stimulation of NO and by scavenging superoxide and may account for cicletanine's protective effects on the cardiovascular and renal systems. That cicletanine increases vascular NO and decreases superoxide and peroxynitrite production is also reported by Szelvassy, et al. (Szelvassy, et al. 2001 J Vascular Res 38:39-46).

These effects of cicletanine should be particularly advantageous for a diabetic individual in view of recent findings on the effects of high glucose on cyclooxygenase-2 (COX-2) and the prostanoid profile in endothelial cells. Cosentino, et al. have shown that high glucose caused PKC-dependent upregulation of inducible COX-2 and eNOS expression and reduced NO release (Cosentino, et al. 2003 Circulation 107:1017-23). The high glucose also resulted in production of ONOO- from NO and superoxide. In another study reported by Mason, et al. (Mason, et al. 2003 J Am Soc Nephrol 14:1358-1373), elevated glucose promoted the formation of reactive oxygen species such as superoxide via activation of several pathways. Thus, cicletanine may act to ameliorate the effects observed under high glucose conditions such as diabetes by its ability to scavenge superoxide and promote formation of NO. Furthermore, cicletanine attenuated glomerular sclerosis in Dahl S rats on a high salt diet suggesting that cicletanine protects the kidney from salt-induced hypertension (Uehara, et al. 1993 Am J Hyperten 6:463-472). Cosentino, et al. also reported a shift in the prostanoid profile towards an overproduction of vasoconstrictor prostanoids with elevated glucose and implicate this shift in diabetes-induced endothelial dysfunction.

Oxatriazoles—

The novel sulfonamide NO donors GEA 3268, (1,2,3,4-oxatriazolium, 3-(3-chloro-2-methylphenyl)-5-[[(4-methoxyphenyl)sulfonyl]amino]-, hydroxide inner salt) and GEA5145, (1,2,3,4-oxatriazolium, 3-(3-chloro-2-methylphenyl)-5-[(methylsulfonyl)amino]-, hydroxide inner salt) are both derivatives of an imine, GEA 3162, that is an NO donor; and sulfonamide GEA 3175, which most probably is an NO donor. It has been suggested that the enzymatic degradation of the sulfonamide moiety has to take place before NO is released.

Inorganic NO Donors—

SNP (sodium nitroprusside, sodium pentacyanonitrosyl ferrate) had been used to treat hypertensive crisis for nearly a century before the mechanism of action of NO was discovered. Together with other commonly used anti-ischemic drugs like glyceryl trinitrate, amyl nitrite and isosorbide dinitrate, it has the disadvantage of consuming organic reduced thiols. The lack of reduced thiols has been implicated in tolerance. SNP is an inorganic complex, in which Fe²⁺ atom is surrounded by 4 cyanides, has a covalent binding to NO, and forms an ion bond to one Na⁺. When the compound becomes decomposed, cyanides are released and this may induce toxicity in long term clinical use. SNP releases NO intracellularly which can lead to problems in the estimation of NO delivery. Though many possible forms of reactive NO derivatives have been discussed, it is somewhat surprising that in vitro SNP-induced relaxation in guinea pig tracheal preparation has been reported to be induced completely via cyclic GMP production.

S-Nitrosothiols (Thionitrates, RSNO)—

S-nitroso-N-acetylpenicillamine (SNAP) is one of the most commonly used NO donors in experimental research since the mid-1990's. In physiological solutions many nitrosothiols rapidly decompose to yield NO. The disadvantage of nitrosothiols is that their half-life can vary from seconds to hours even at a pH of 7.4, and this is dependent on the buffer used. In physiological buffers, many of the RSNOs become decomposed rapidly to yield disulfide and NO.

Sydnonimines—

SIN-1 is the active metabolite of the antianginal prodrug molsidomine (N-ethoxycarbonyl-3-morpholinosydnonimine), these two compounds are sydnonimines that are also mesoionic heterocycles. Liver metabolism needs to convert molsidomine it into its active form. SIN-1 is a potent vasorelaxant and an antiplatelet agent causing spontaneous, extracellular release of NO. SIN-1 can activate sGC independently of thiol groups. SIN-1 can rapidly and non-enzymatically hydrolyze into SIN-1A when there are traces of oxygen present, it donates NO and spontaneously turns into NO-deficient SIN-1C. SIN-1C prevents human neutrophil degranulation in a concentration-dependent manner and can reduce Ca²⁺ increase, a property which is common to SIN-1. SIN-1 has been shown to release NO, ONOO- and O²⁻.

NO Inducers—

Various drugs and compositions have been shown to up-regulate endogenous NO release by inducing NOS expression. For example, Hauser et al. 1996 Am J Physiol 271:H2529-35), reported that endotoxin (lipopolysaccharide, LPS)-induced hypotension is, in part, mediated via induction of NOS, release of nitric oxide, and suppression of vascular reactivity (vasoplegia).

### Calcium Channel Blockers

Calcium channel blockers act by blocking the entry of calcium into muscle cells of heart and arteries so that the contraction of the heart decreases and the arteries dilate. With the dilation of the arteries, arterial pressure is reduced so that it is easier for the heart to pump blood. This also reduces the heart's oxygen requirement. Calcium channel blockers are useful for treating angina. Due to blood pressure lowering effects, calcium channel blockers are also useful to treat high blood pressure. Because they slow the heart rate, calcium channel blockers may be used to treat rapid heart rhythms such as atrial fibrillation. Calcium channel blockers are also administered to patients after a heart attack and may be helpful in treatment of arteriosclerosis.

Examples of calcium channel blockers include diltiazem malate, amlodipine bensylate, verapamil hydrochloride, diltiazem hydrochloride, nifedipine, felodipine, nisoldipine, isradipine, nimodipine, nicardipine hydrochloride, bepridil hydrochloride, and mibefradil di-hydrochloride. The scope of the present invention includes all those calcium channel blockers now known and all those calcium channel blockers to be discovered in the future.

Preferred calcium channel blockers comprise amlodipine, diltiazem, isradipine, nicardipine, nifedipine, nimodipine, nisoldipine, nitrendipine, and verapamil, or, e.g. dependent on the specific calcium channel blockers, a pharmaceutically acceptable salt thereof. Especially preferred is amlodipine or a pharmaceutically acceptable salt thereof, especially the besylate.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having at least one acid group (for example COOH) can also form salts with bases. Corresponding internal salts may furthermore be formed, if a compound of formula comprises e.g., both a carboxy and an amino group.

Preferred salts of corresponding calcium channel blockers are amlodipine besylate, diltiazem hydrochloride, fendiline hydrochloride, flunarizine di-hydrochloride, gallopamil hydrochloride, mibefradil di-hydrochloride, nicardipine hydrochloride, lercanidipine and verapamil hydrochloride.

In accordance with one preferred embodiment of the present combination therapy, cicletanine is administered together with the second generation calcium antagonist, amlodipine. The combination may administered in a sustained release dosage form. Because amlodipine is a long acting compound it may not warrant sustained release; however, where cicletanine is dosed two or more times daily, then in accordance with one embodiment, the cicletanine may be administered in sustained release form, along with immediate release amlodipine. Preferably, the combination dosage and release form is optimized for the treatment of hypertensive patients. Most preferably, the oral combination is administered once daily.

### ACE Inhibitors

Angiotensin converting enzyme (ACE) inhibitors are compounds that inhibit the action of angiotensin converting enzyme, which converts angiotensin I to angiotensin II. ACE inhibitors have individually been shown to be somewhat effective in the treatment of cardiac disease, such as congestive heart failure, hypertension, asymptomatic left ventricular dysfunction, or acute myocardial infarction.

A number of ACE inhibitors are known and available. These compounds include inter alfa lisinopril (Zestril®; Prinivil®), enalapril maleate (Innovace®; Vasotec®), quinapril (Accupril®), ramipril (Tritace®; Altace®), benazepril (Lotensin®), captopril (Capoten®), cilazapril (Vascace®), fosinopril (Staril®; Monopril®), imidapril hydrochloride (Tanatril®), moexipril hydrochloride (Perdix®; Univasc®), trandolapril (Gopten®; Odrik®; Mavik®), and perindopril (Coversyl®; Aceon®). The scope of the present invention includes all those ACE inhibitors now known and all those ACE inhibitors to be discovered in the future.

In accordance with one preferred embodiment of the present combination therapy, cicletanine is administered together with an ACE inhibitor. Preferably the combination is administered in a once-daily oral dosage form. Preferably, the combination is optimized for treatment of hypertension in patients with and without type 2 diabetes mellitus. Some of the major endpoints of such a study would be effects on blood pressure, left ventricular function, insulin sensitivity, and renal functions.

### Angiotensin II Receptor Antagonists

Angiotensin II receptor antagonists (blockers; ARB's), lower both systolic and diastolic blood pressure by blocking one of four receptors with which angiotensin II can interact to effect cellular change. Examples of angiotensin II receptor antagonists include losartan potassium, valsartan, irbesartan, candesartan cliexetil, telmisartan, eprosartan mesylate, and olmesartan medoxomil. Angiotensin II receptor antagonists in combination with a diuretic are also available and include losartan potassium/hydrochlorothiazide, valsartan/hydrochlorothiazide, irbesartan/hydrochlorothiazide, candesartan cilexetil/hydrochlorothiazide, and telmisartan/hydrochlorothiazide. The scope of the present invention includes all those angiotensin receptor antagonists now known and all those angiotensin receptor antagonists to be discovered in the future.

### Diuretics

Individual diuretics increase urine volume. One mechanism is by inhibiting reabsorption of liquids in a specific segment of nephrons, e.g., proximal tubule, loop of Henle, or distal tubule. For example, a loop diuretic inhibits reabsorption in the loop of Henle. Examples of diuretics commonly used for treating hypertension include hydrochlorothiazide, chlorthalidone, bendroflumethazide, benazepril, enalapril, and trandolapril. The scope of the present invention includes all those diuretics now known and all those diuretics to be discovered in the future.

### Beta Blockers

Beta blockers prevent the binding of adrenaline to the body's beta receptors which blocks the “fight or flight” response. Beta receptors are found throughout the body, including the heart, lung, arteries and brain. Beta blockers slow down the nerve impulses that travel through the heart. Consequently, the heart needs less blood and oxygen. Heart rate and force of heart contractions are decreased.

There are two types of beta receptors, beta 1 and beta 2 that are commonly targeted in hypertension therapy. Beta 1 receptors are associated with heart rate and strength of heart beat and some beta blockers selectively block beta 1 more than beta 2. Beta blockers are used to treat a wide variety of conditions including high blood pressure, congestive heart failure, tachycardia, heart arrhythmias, angina, migraines, prevention of a second heart attack, tremor, alcohol withdrawal, anxiety, and glaucoma.

A number of beta blockers are known which include atenolol, metoprolol succinate, metoprolol tartrate, propranolol hydrochloride, nadolol, acebutolol hydrochloride, bisoprolol fumarate, pindolol, betaxolol hydrochloride, penbutolol sulfate, timolol maleate, carteolol hydrochloride, esmolol hydrochloride. Beta blockers, generally, are compounds that block beta receptors found throughout the body. The scope of the present invention includes all those beta blockers now known and all those beta blockers to be discovered in the future.

### Aldosterone Antagonists

Aldosterone is a mineralocorticoid steroid hormone which acts on the kidney promoting the reabsorption of sodium ions (Na⁺) into the blood. Water follows the salt, helping maintain normal blood pressure. Aldosterone has the potential to cause edema through sodium and water retention. Aldosterone antagonists inhibit the action of aldosterone and have shown significant benefits for patients suffering from congestive heart failure, hypertension, and microalbuminuria.

A number of aldosterone antagonists are known including sprironolactone and eplerenone (Inspra®). Aldosterone antagonists, generally, are compounds that block the action of aldosterone throughout the body. The scope of the present invention includes all those aldosterone antagonists now known and those aldosterone antagonists to be discovered in the future.

Other classes of antihypertensive agents that are envisioned in combination with cicletanine are: endothelin antagonists, urotensin antagonists, vasopeptidase inhibitors, neutral endopeptidase inhibitors, hydroxymethylglutaryl-CoA (HMG-CoA) reductase inhibitors, vasopressin antagonists, and T-type calcium channel antagonists.

### Endothelin Antagonists

Endothelin-1 (ET-1) is a potent vasoconstrictor, and thus its role in the development and/or maintenance of hypertension has been studied extensively. ET-1, the predominant isoform of the endothelin peptide family, regulates vasoconstriction and cell proliferation in tissues both within and outside the cardiovascular system through activation of protein-coupled ETA or ETB receptors. The endothelin system has been implicated in the pathogenesis of arterial hypertension and renal disorders. Plasma endothelin also appears to be greater in obese individuals, particularly obese hypertensives. Blood vessel endothelin expression and cardiac levels of ET-1-like immunoreactivity have been shown to be increased in various animal models of hypertension. Renal prepro-ET-1 mRNA levels are also increased in DOCA-salt hypertensive animals and endothelin production from cultured endothelial cells is upregulated in hypertensive rats. Both ETA and ETB receptors have been shown to be reduced in mesenteric vessels of spontaneously hypertensive rats. There are a number of experimental studies demonstrating that direct and indirect endothelin-antagonists can have beneficial effects in hypertension.

Administration of the endothelin-converting enzyme inhibitor, phosphoramidon, or ET-receptor antagonists (e.g., bosentan) have been shown to reduce blood pressure in a number of different hypertensive rat models.

### Neutral Endopeptidase Inhibitors

Since angiotensin II is an established target of pharmacologic interventions, there is an increasing interest in the biological effects and metabolism of other vasoactive peptides, such as atrial natriuretic peptide (ANP) and ET. Exogenous administration of the vasodilatory and natriuretic ANP and of its analogues improved hemodynamics and renal function in cardiovascular disease, including congestive heart failure. Promising results have been obtained in animal experiments and initial human clinical studies concerning hemodynamics and kidney function with inhibition of ANP metabolism by inhibitors of neutral endopeptidase (NEP). In further clinical studies, moderately relevant effects of acute intravenous or oral NEP inhibition were observed, but these effects were blunted with acute drug administration. There is increasing evidence the NEP inhibitors, such as candoxatril and ecadotril, expected to exhibit vasodilatory activity at least at certain doses in certain clinical situations, even induce vasoconstriction. An explanation for the ineffectiveness of NEPs in reducing blood pressure when used alone may lie in the effect of the role of NEP in the metabolism of other peptides besides ANP. In addition to ANP and other natriuretic peptides, NEP also metabolizes the vasoactive peptides ET-1, angiotensin II, and bradykinin.

### Vasopeptidase Inhibitors

Vasopeptidase inhibition is a novel efficacious strategy for treating cardiovascular disorders, including hypertension and heart failure, that may offer advantages over currently available therapies. Vasopeptidase inhibitors are single molecules that simultaneously inhibit two key enzymes involved in the regulation of cardiovascular function, NEP and ACE. Simultaneous inhibition of NEP and ACE increases natriuretic and vasodilatory peptides (including ANP), brain natriuretic peptide of myocardial cell origin, and C-type natriuretic peptide of endothelial origin. This inhibition also increases the half-life of other vasodilator peptides, including bradykinin and adrenomedullin. By simultaneously inhibiting the renin-angiotensin-aldosterone system and potentiating the natriuretic peptide system, vasopeptidase inhibitors reduce vasoconstriction and enhance vasodilation, thereby decreasing vascular tone and lowering blood pressure. Omapatrilat, a heterocyclic dipeptide mimetic, is the first vasopeptidase inhibitor to reach advanced clinical trials in the United States. Unlike ACE inhibitors, omapatrilat demonstrates antihypertensive efficacy in low-, normal-, and high-renin animal models. Unlike NEP inhibitors, omapatrilat provides a potent and sustained antihypertensive effect in spontaneously hypertensive rats, a model of human essential hypertension. In animal models of heart failure, omapatrilat is more effective than ACE inhibition in improving cardiac performance and ventricular remodeling and prolonging survival. Omapatrilat effectively reduces blood pressure, provides target organ protection, and reduces morbidity and mortality from cardiovascular events in animal models. Human studies with omapatrilat (Vanlev, Bristol-Myers Squibb), administered orally once daily, have demonstrated a dose-dependent reduction of systolic and diastolic blood pressure, regardless of age, race, or gender. Its ability to decrease systolic blood pressure is especially notable, since evidence suggests that systolic blood pressure is a better predictor than diastolic blood pressure of stroke, heart attack, and death. Omapatrilat appears to be a safe, well-tolerated, effective hypertensive agent in humans, and it has the potential to be an effective, broad-spectrum antihypertensive agent. Adverse effects are comparable to those of currently available antihypertensive agents. Another vasopeptidase inhibitor that is currently under clinical development is the agent sampatrilat (Chiron).

### HMG-CoA Reductase Inhibitors

HMG-CoA reductase inhibitors (e.g., statins) are increasingly being used to treat high cholesterol levels and have been shown to prevent heart attacks and strokes. Many individuals with high cholesterol also have high blood pressure, so the effect of the statins on blood pressure is of great interest. Certain HMG-CoA reductase inhibitors may cause vasodilation by restoring endothelial dysfunction, which frequently accompanies hypertension and hypercholesterolemia. There have also been reports of a synergistic effect on vasodilation between ACE inhibitors and statins. Several studies have found that a blood pressure reduction is associated with the use of statins, but conclusive evidence from controlled trials is lacking. In a recent clinical study in individuals with moderate hypercholesterolemia and untreated hypertension, the HMG-CoA reductase inhibitor pravastatin (20 to 40 mg/day, 16 weeks) decreased total (6.29 to 5.28 mmol/L) and low-density lipoprotein (4.31 to 3.22 mmol/L) cholesterol, systolic and diastolic blood pressure (149/97 to 131/91), and pulse pressure. In this same study, circulating ET-1 levels were decreased by pretreatment with pravastatin. In conclusion, clinical studies have demonstrated that a specific statin, pravastatin, decreases systolic, diastolic, and pulse pressures in persons with moderate hypercholesterolemia and hypertension.

### Vasopressin Antagonists

It has long been known that the hormone vasopressin plays an important role in peripheral vasoconstriction, hypertension, and in several disease conditions with dilutional hyponatremia in edematous disorders, such as congestive heart failure, liver cirrhosis, syndrome of inappropriate secretion of antidiuretic hormone, and nephrotic syndrome. These effects of vasopressin are mediated through vascular (V1a) and renal (V2) receptors. A series of orally active nonpeptide antagonists against the vasopressin receptor subtypes have recently been synthesized and are now under intensive examination. Nonpeptide V1a-receptor antagonists, OPC21268 and SR49059, nonpeptide V2-receptor-specific antagonists, SR121463A and VPA985, and combined V1a/V2-receptor antagonists, OPC31260 and YM087, are currently available.

### T-Type Calcium Ion Channel Antagonists

Recent clinical trials have been conducted with a new class of calcium channel antagonists that selectively block T-type voltage-gated plasma membrane calcium channels in vascular smooth muscle. The prototypical member of this group is the agent mibefradil (Roche), which is 10 to 50 times more selective for blocking T-type than L-type calcium channels. This drug is structurally and pharmacologically different from traditional calcium antagonists. It does not produce negative inotropic effects at therapeutic concentrations and is not associated with reflex activation of neurohormonal and sympathetic systems. In clinical studies of hypertension, mibefradil (50 and 100 mg/day) reduced trough sitting diastolic and systolic blood pressure in a dose-related manner. Dosages exceeding 100 mg/day generally did not result in significantly greater efficacy, but were associated with a higher frequency of adverse events. No first-dose hypotensive phenomenon was observed. Mibefradil has antiischemic properties resulting from dilation of coronary and peripheral vascular smooth muscle, and a slight reduction in heart rate. Mibefradil (Posicor®) was approved by the FDA in June 1997 for the treatment of hypertension and angina, but was withdrawn from the market in 1998 because of severe drug interactions. Since the effects of this type of calcium channel blocker were so profound on hypertension, studies with other selective T-type calcium channel antagonists have continued.

### Urotensin-II Antagonists

Recent discoveries have identified Urotensin-II (U-II) as an important regulator of the cardiovascular system, working to constrict arteries and possibly to increase blood pressure in response to exercise and stress. It was found that U-II constricts arteries more mildly and for a longer period than other chemicals known for similar effects on blood pressure. The potency of vasoconstriction of U-II is an order of magnitude greater than that of ET-1, making human U-II the most potent mammalian vasoconstrictor identified to date. In vivo, human U-II markedly increases total peripheral resistance in anesthetized nonhuman primates, a response associated with profound cardiac contractile dysfunction. These effects are mediated by U-II binding to receptors in the brainstem, heart, and in major blood vessels, including the pulmonary artery, which supplies blood to the lungs, and the aorta, the major vessel leading from the heart.

### PPAR Agonists

Peroxisome proliferator-activated receptors (PPARs) are a family of ligand-activated nuclear hormone receptors belonging to the steroid receptor super-family that regulate lipid and carbohydrate metabolism in response to extracellular fatty acids and their metabolites. They may be important in the regulation of fat storage, besides having a potential role in insulin resistance syndrome. They also may have relevance in understanding the cause of common clinical conditions such as type 2 diabetes mellitus, cellular growth and neoplasia, and in the development of drugs for treating such conditions. Three types of receptors were identified: PPAR alpha, gamma and delta. Whereas PPAR alpha is a regulator of fatty acid catabolism in the liver PPAR gamma plays a key role in adipogenesis. The use of synthetic PPAR ligands has demonstrated the involvement of these receptors in the regulation of lipid and glucose homeostasis and today PPARs are established molecular targets for the treatment of type 2 diabetes and cardiovascular disease. The librate family of lipid lowering agents binds to the alpha isoform and the glitazone family of insulin sensitizers binds to the gamma isoform of PPARs.

### Oral Antidiabetics

Sulfonureas—

The sulfonylurea group has dominated oral antidiabetic treatment for years. They primarily increase insulin secretion. Their action is initiated by binding to and closing a specific sulfonylurea receptor (an ATP-sensitive K⁺ channel) on pancreatic β-cells. This closure decreases K⁺ influx, leading to depolarization of the membrane and activation of a voltage-dependent Ca²⁺ channel. The resulting increased Ca²⁺ flux into the β-cell, activates a cytoskeletal system that causes translocation of insulin to the cell surface and its extrusion by exocytosis.

The proximal step in this sulfonylurea signal transduction is the binding to (and closure) of high-affinity protein receptors in the O-cell membrane. There are both high and low-affinity sulfonylurea receptor populations. Sulfonylurea binding to the high-affinity sites affects primarily K⁺(ATP) channel activity, while interaction with the low-affinity sites inhibits both Na⁺/K⁺-ATPase and K(ATP) channel activities. The potent second-generation sulfonylureas, glyburide and glipizide, are able to saturate receptors in low nanomolar concentration ranges, whereas older, first-generation drugs bind to and saturate receptors in micromolar ranges.

There is a synergy between the action of glucose and that of the sulfonylureas: sulfonylureas are better effectors of insulin secretion in the presence of glucose. For that reason, the higher the level of plasma glucose at the time of initiation of sulfonylurea treatment, the greater the reduction of hyperglycemia.

Exposure of perfused rat hearts to the second-generation sulfonylurea glyburide leads to a dramatic increase in glycolytic flux and lactate production. When insulin is included in the buffer, the response to glyburide is significantly increased. (Similarly, glyburide potentiates the metabolic effects of insulin.) Because glyburide does not promote glycogenolysis, this increase in glycolytic flux is caused solely by a rise in glucose utilization. Since the drug does not alter oxygen consumption, the contribution of glucose to overall ATP production rises while that of fatty acids falls. These metabolic changes aid the heart in resisting ischemic insults.

Insulin, on the other hand, is released by the pancreas into the portal vein, where the resultant hyperinsulinemia suppresses hepatic glucose production and the elevated level of arterial insulin enhances muscle glucose uptake, leading to a reduction in postprandial plasma glucose levels.

The initial hypoglycemic effect of sulfonylureas results from increased circulating insulin levels secondary to the stimulation of insulin release from pancreatic β-cells and, perhaps to a lesser extent, from a reduction in its hepatic clearance. Unfortunately, these initial increases in plasma insulin levels and β-cell responses to oral glucose are not sustained during chronic sulfonylurea therapy. After a few months, plasma insulin levels decline to those that existed before treatment, even though reduced glucose levels are maintained. Because of downregulation of β-cell membrane receptors for sulfonylurea, its chronic use results in a reduction in the insulin stimulation usually recorded following acute administration of these drugs. More globally, impairment of even proinsulin biosynthesis and, in some instances, inhibition of nutrient-stimulated insulin secretion may follow chronic (greater than several months) administration of any of the sulfonylureas. (However, the initial view that the proinsulin/insulin ratio is reduced by sulfonylurea treatment seems unlikely in light of recent research.). If chronic sulfonylurea therapy is discontinued, a more sensitive pancreatic β-cell responsiveness to acute administration of the drug is restored.

It is probable that this long-term sulfonylurea failure results from chronically lowered plasma glucose levels (and a resulting feedback reduction of sulfonylurea stimulation); it does, however, lead to a diminishment of the vicious hyperglycemia-hyperinsulinemia cycle of glucose toxicity. As a result, the sulfonylureas reduce nonenzymatic glycation of cellular proteins and the association of the latter with an increased generation of advanced glycation end products (AGEs), and improve insulin sensitivity at the target tissues. But, it should be kept in mind that one of these cellular proteins is insulin, which is readily glycated within pancreatic β-cells and under these conditions, when it is secreted it presumably is now ineffective as a ligand.

It has been suggested that sulfonylureas may have a direct effect in reducing insulin resistance on peripheral tissues. However, most investigators believe that whatever small improvement in insulin action is observed during sulfonylurea treatment is indirect, possibly explained (as above) by the lessening of glucose toxicity and/or by decreasing the amount of ineffective, glycated insulin.

When sulfonylurea treatment is compared with insulin treatment it is found that: (1) treatment with sulfonylurea or insulin results in equal improvement in glycemia and insulin sensitivity, (2) the levels of proinsulin and plasminogen activator inhibitor-1 (PAI-1) antigen and its activity are higher with sulfonylurea, and (3) there are no differences in lipid concentrations between therapies.

Type 2 diabetes mellitus is part of a complicated metabolic-cardiovascular pathophysiologic cluster alternately referred to as the insulin resistance syndrome, Reaven's syndrome, the metabolic syndrome or syndrome X. Since the macrovascular coronary artery disease associated with insulin resistance and type 2 diabetes is the major cause of death in the latter, it is desirable that any hypoglycemic agent favorably influences known cardiovascular risk factors. But the results in this area have been only mildly encouraging. This invention will add a cardiovascular risk reduction dimension to sulfonylurea therapy.

Sulfonylureas have been reported to have a neutral or just slightly beneficial effect on plasma lipid levels: plasma triglyceride levels decrease modestly in some studies. This hypolipidemic effect probably results from both a direct effect of sulfonylurea on the metabolism of very-low-density lipoprotein (VLDL) and an indirect effect of sulfonylurea secondary to its reduction of plasma glucose levels.

The formulations of this invention provide appropriate therapeutic levels of a sulfonylurea and will enhance and/or extend the beneficial effect of the sulfonylureas upon plasma lipids, coagulopathy and microvascular permeability by additionally lowering the blood pressure.

The most frequent adverse effect associated with sulfonylurea therapy is weight gain, which is also implicated as a cause of secondary drug failure. The side effects of the various sulfonylureas may vary among the members of the family.

Sulfonylureas frequently: (1) stimulate renal renin release; (2) inhibit renal carnitine resorption; (3) increase PAI-1; and (4) increase insulin resistance.

Renal effects from treatment with the sulfonylureas can be detrimental. Because the sulfonylureas are K_{ATP} blockers they are diuretics although, fortunately, they do not produce kaliuresis. They may stimulate renin secretion from the kidney, initiating a cascade to angiotensin II in the vascular endothelium that results in vasoconstriction and elevated blood pressure. Therefore, the therapeutic combination of the present invention will be beneficial to controlling the renal side effects of sulfonureas.

The most discussed, important adverse effect of chronic sulfonylureas use is long lasting, significant hypoglycemia. The latter may lead to permanent neurological damage or even death, and is most commonly seen in elderly subjects who are exposed to some intercurrent event (e.g., acute energy deprivation) or to drug interactions (e.g., aspirin, alcohol). Long-lasting hypoglycemia is more common with the longer-acting sulfonylureas glyburide and chlorpropamide. For this reason sulfonylurea therapy should be maintained at the lowest possible dose. By complementing and efficiently optimizing the therapeutic action of sulfonylurea, the formulations of this invention permit the use of minimal doses of sulfonylureas, thereby lowering the risks of sulfonylurea therapy, including hypoglycemia.

As our population ages and as the prevalence of ‘couch potatoes’ rises, the danger of sulfonylurea hypoglycemia continually increases. The formulations of this invention are of increasing importance, because they permit clinical reductions in sulfonylurea dose levels.

Sulfonylureas are divided into first-generation and second-generation drugs. First-generation sulfonylureas have a lower binding affinity to the sulfonylurea receptor and require higher doses than second-generation sulfonylureas. Generally, therapy is initiated at the lowest effective dose and titrated upward every 1 to 4 weeks until a fasting plasma glucose level of 110 to 140 mg/dL is achieved. Most (75%) of the hypoglycemic action of the sulfonylurea occurs with a daily dose that is half of the maximally effective dose. If no hypoglycemic effect is observed with half of the maximally effective dose, it is unlikely that further dose increases will have a clinically significant effect on blood glucose level.

In summary, sulfonylureas are effective glucose-lowering drugs that work by stimulating insulin secretion. They have a beneficial effect on diabetic microangiopathy, but no appreciable beneficial effect on diabetic macroangiopathy. Weight gain is common with their use. Sulfonylureas may cause hypoglycemia, which can be severe, even fatal. They may reduce platelet aggregation and slightly increase fibrinolysis, perhaps indirectly. They have no direct effect on plasma lipids. They inhibit renal resorption of carnitine and may stimulate renal renin secretion. The sulfonylureas, especially generics, are inexpensive. Sulfonylurea dosage can be minimized, therapeutic effect maximized, safety improved and the scope of beneficial effects broadened in progressive insulin resistance, insulin resistance syndrome and type 2 diabetes when delivered in the formulations of this invention.

Biguanides (Metformin)—

Metformin (Glucophage®) has a unique mechanism of action and controls glycemia in both obese and normal-weight, type 2 diabetes patients without inducing hypoglycemia, insulin stimulation or hyperinsulinemia. It prevents the desensitization of human pancreatic islets usually induced by hyperglycemia and has no significant effect on the secretion of glucagon or somatostatin. As a result it lowers both fasting and postprandial glucose and HbAlc levels. It also improves the lipid profile.

Glucose levels are reduced during metformin therapy secondary to reduced hepatic glucose output from inhibition of gluconeogenesis and glycogenolysis. To a lesser degree it increases insulin action in peripheral tissues.

Metformin enhances the sensitivity of both hepatic and peripheral tissues (primarily muscle) to insulin as well as inhibiting hepatic gluconeogenesis and hepatic glycogenolysis. This decline in basal hepatic glucose production is correlated with a reduction in fasting plasma glucose levels. Its enhancement of muscle insulin sensitivity is both direct and indirect. Improved insulin sensitivity in muscle from metformin is derived from multiple events, including increased insulin receptor tyrosine kinase activity, augmented numbers and activity of GLUT4 transporters, and enhanced glycogen synthesis. However, the primary receptor through which metformin exerts its effects in muscle and in the liver is as yet unknown. In metformin-treated patients both fasting and postprandial insulin levels consistently decrease, reflecting a normal response of the pancreas to enhanced insulin sensitivity.

Metformin has a mean bioavailability of 50-60%. It is eliminated primarily by renal filtration and secretion and has a half-life of approximately 6 hours in patients with type 2 diabetes; its half-life is prolonged in patients with renal impairment. It has no effect in the absence of insulin. Metformin is as effective as the sulfonylureas in treating patients with type 2 diabetes, but has a more prominent postprandial effect than either the sulfonylureas or insulin. It is therefore most useful in managing patients with poorly controlled postprandial hyperglycemia and in obese or dyslipidemic patients; in contrast, the sulfonylureas or insulin are more effective in managing patients with poorly controlled fasting hyperglycemia.

Metformin is absorbed mainly from the small intestine. It is stable, does not bind to plasma proteins, and is excreted unchanged in the urine. It has a half-life of 1.3 to 4.5 hours. The maximum recommended daily dose of metformin is 3 g, taken in three doses with meals.

When used as monotherapy, metformin clinically decreases plasma triglyceride and low-density lipoprotein (LDL) cholesterol levels by 10% to 15%, reduces postprandial hyperlipidemia, decreases plasma free fatty acid levels, and free fatty acid oxidation. Metformin reduces triglyceride levels in non-diabetic patients with hypertriglyceridemia. HDL cholesterol levels either do not change or increase slightly after metformin therapy. By reducing hyperinsulinemia, metformin improves levels of plasminogen activator inhibitor (PAI-1) and thus improves fibrinolysis in insulin resistance patients with or without diabetes. Weight gain does not occur in patients with type 2 diabetes who receive metformin; in fact, most studies show modest weight loss (2 to 3 kg) during the first 6 months of treatment. In one 1-year randomized, double blind trial, 457 non-diabetic patients with android (abdominal) obesity, metformin caused significant weight loss.

Metformin reduces blood pressure, improves blood flow rheology and inhibits platelet aggregation. The latter is also an effect of prostacyclins, and cicletanine which increases endogenous prostacyclin. See e.g., Arch Mal Coeur Vaiss. 1989 November; 82 Spec No 4:11-4.

These beneficial effects of metformin on various elements of the insulin resistance syndrome help define its usefulness in the treatment of insulin resistance and type 2 diabetes. These useful effects are enhanced when metformin is combined with components of this invention (e.g. cicletanine). The latter is envisioned to increase its effectiveness and efficiency, improve its safety and expand the arena of its medical benefit. On the other hand, metformin in combination with cicletanine is envisioned to allow reduction in the dose of the latter to achieve the same antihypertensive effect.

Metformin reduces measurable levels of plasma triglycerides and LDL cholesterol and is the only oral, monotherapy, antidiabetic agent that has the potential to reduce macrovascular complications, although this favorable effect is attenuated by its tendency to increase homocysteine levels. Likewise, it is the only oral hypoglycemic drug wherein most patients treated lose weight or fail to gain weight.

This invention introduces a strategy to increase the safety and efficiency of metformin in suppressing recognized risk factors, thus slowing the progression of disease by extending both the duration and the breadth of metformin's therapeutic value. The strategy of this invention will increase the number of patients by whom metformin can be used at reduced dose levels, thereby avoiding, delaying and lessening metformin's adverse effects.

Gastrointestinal side effects (diarrhea, nausea, abdominal pain, and metallic taste in decreasing order) are the most common adverse events, occurring in 20% to 30% of patients. These side effects usually are mild and transient and can be minimized by slow titration. If side effects occur during titration, they can be eliminated by reducing the dose by administering metformin in the combination of the present invention.

Meglitinides and Phenylalanine Derivatives—

Meglitinides, such as repaglinide, are derived from the non-sulfonylurea part of the glyburide molecule and nateglinide is derived from D-phenylalanine. Both repaglinide and nateglinide bind competitively to the sulfonylurea receptor of the pancreatic β-cell and stimulate insulin release by inhibiting K_{ATP} channels in the β-cells. The relative potency of inhibition of K_{ATP} channels is repaglinide>glyburide>nateglinide. Nateglinide exhibits rapid inhibition and reversal of inhibition of the K_{ATP} channel.

The plasma half-life of these drugs (50-60 min) is much shorter than that of glyburide (4-11 h). Repaglinide and nateglinide are absorbed rapidly, stimulate insulin release within a few minutes, and are quickly metabolized. Repaglinide is excreted by the liver and nateglinide is excreted by the kidneys.

Insulin secretion is more rapid in response to nateglinide than in response to repaglinide. If nateglinide is taken before a meal, insulin becomes available during and after the meal, significantly reducing postprandial hyperglycemia without the danger of hypoglycemia between meals. Nateglinide, therefore, may potentially replace the absent Phase 1 insulin secretion in patients with type 2 diabetes.

The meglitinides and D-phenylalanine derivatives, classified as “prandial glucose regulators,” must be taken before each meal. The dosage can be adjusted according to the amount of carbohydrate consumed. These drugs are especially useful when metformin is contraindicated (e.g., in patients with creatinine clearance <50 ml/min). Treatment can be combined with other OADs as well as with cicletanine.

As a result of the rapidity of their insulin-releasing action, repaglinide and nateglinide are more effective in reducing postprandial hyperglycemia and pose a lower hypoglycemia risk than sulfonylureas such as glyburide.

α-Glucosidase Inhibitors—

The α-glucosidase inhibitors (e.g., acarbose, miglitol, and voglibose) reduce the small intestinal absorption of starch, dextrin, and disaccharides by competitively inhibiting the action of the intestinal brush border enzyme, α-glucosidase. α-Glucosidase is responsible for the generation of monosaccharides, so that inhibition of α-glucosidase, which is the final step in carbohydrate transfer across the small intestinal mucosa, slows down the absorption of carbohydrates.

These drugs are used for the treatment of patients with type 2 diabetes who are inadequately controlled by diet or other oral antidiabetic drugs. Clinical trials of α-glucosidase inhibitors show decreases in postprandial glucose levels, especially when taken at the start of a meal, as well as decreases in glycosylated hemoglobin (HbAlc) of 0.5-1%. It has been reported that miglitol reduces HbAlc less effectively than glyburide (glibenclamide) and also causes more alimentary side effects. Miglitol, which must be taken with each meal, has little effect on fasting blood glucose concentrations but blunts postprandial glucose increases at lower postprandial insulin concentrations than those observed with sulfonylureas. Unlike glyburide, miglitol is not associated with hypoglycemia, hyperinsulinism, or weight gain.

The combination of acarbose or miglitol with, for example, cicletanine is envisioned to achieve the therapeutic effects of the individual agents in the composition of the present invention at lower doses that when administered individually, therefore reducing the incidence of side effects.

### Formulations and Treatment Regimens

For oral and bucchal administration, a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, stearic acid and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents coloring agents, emulsifying agents and/or suspending agents, as well as such diluents such as water, ethanol, propylene glycol, glycerin and various like combinations thereof.

For purposes of parenteral administration, solutions in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easter, Pa., 15^{th} Edition (1975).

In one embodiment of the present invention, a therapeutically effective amount of each component may be administered simultaneously or sequentially and in any order. The corresponding active ingredient or a pharmaceutically acceptable salt thereof may also be used in form of a hydrate or include other solvents used for crystallization. The pharmaceutical compositions according to the invention can be prepared in a manner known per se and are those suitable for enteral, such as oral or rectal, and parenteral administration to mammals (warm-blooded animals), including man, comprising a therapeutically effective amount of the pharmacologically active compound, alone or in combination with one or more pharmaceutically acceptable carriers, especially suitable for enteral or parenteral application.

The novel pharmaceutical preparations contain, for example, from about 10% to about 80%, preferably from about 20% to about 60%, of the active ingredient. In one aspect, pharmaceutical preparations according to the invention for enteral administration are, for example, those in unit dose forms, such as film-coated tablets, tablets, or capsules. These are prepared in a manner known per se, for example by means of conventional mixing, granulating, or film-coating. Thus, pharmaceutical preparations for oral use can be obtained by combining the active ingredient with solid carriers, if desired granulating a mixture obtained, and processing the mixture or granules, if desired or necessary, after addition of suitable excipients to give tablets or film-coated tablet cores.

In another aspect, novel pharmaceutical preparations for parenteral administration contain, for example, from about 10% to about 80%, preferably from about 20% to about 60%, of the active ingredient. These novel pharmaceutical preparations include liquid formulations for injection, suppositories or ampoules. These are prepared in a manner known per se, for example by means of conventional mixing, dissolving or lyophilizing processes.

### Treatment of Metabolic Syndrome

Cicletanine, due to its multiple therapeutic effects, may also be used in accordance with preferred embodiments of the present invention as a treatment for metabolic syndrome (sometimes also known as “pre-diabetes” or “syndrome X”). The National Cholesterol Education Program (NCEP) at the NIH lists the following as “factors that are generally accepted as being characteristic of [metabolic] syndrome” (Third Report of the Expert Panel on Detection, Evaluation, and Treatment of High Blood Cholesterol in Adults (Adult Treatment Panel III; also known as ATP III). Nov. 19, 2002. National Heart, Lung and Blood Institute (NHLBI), National Institutes of Health): abdominal obesity; atherogenic dyslipidemia; raised blood pressure; insulin resistance±glucose intolerance; prothrombotic state; proinflammatory state.

For purposes, of diagnosis, the metabolic syndrome is identified by the presence of three or more of the components listed in Table 4 below:

TABLE 4
Clinical Identification of the Metabolic Syndrome*

| Risk Factor | Defining Level |
|---|---|
| Abdominal Obesity | Men >102 cm (>40″); Women >88 cm (>35″) |
| Waist Circumference^{†} | |
| Triglycerides | ≧150 mg/dl |
| HDL cholesterol | Men <40 mg/dl; Women <50 mg/dL |
| Blood pressure | ≧130/85 mmHg |
| Fasting glucose | ≧110 mg/dl |

*The ATP III panel did not find adequate evidence to recommend routine measurement of insulin resistance (e.g., plasma insulin), proinflammatory state (e.g., high-sensitivity C-reactive protein), or prothrombotic state (e.g., fibrinogen or PAI-1) in the diagnosis of the metabolic syndrome.
^{†}Some male persons can develop multiple metabolic risk factors when the waist circumference is only marginally increased, e.g., 94-102 cm (37″-39″). Such persons may have a strong genetic contribution to insulin resistance. They should benefit from changes in life habits, similarly to men with categorical increases in waist circumference.

Cicletanine as a combination therapy with another drug (such as an ACE inhibitor or an angiotensin II receptor antagonist, or an OAD or a Lipid-lowering agent), holds promise addressing these five factors.

### Abdominal Obesity

For example, abdominal obesity, and perhaps obesity in general, is likely to be one step upstream on the causal chain of metabolic syndrome from the point of action of cicletanine. In a recent review article (Hall J. E. 2003 Hypertension 41:625-33), the author charts an accepted view of the role of obesity in hypertension.

Obesity increases renal sodium reabsorption and impairs pressure natriuresis by activation of the renin-angiotensin and sympathetic nervous systems and by altered intrarenal physical forces. Chronic obesity also causes marked structural changes in the kidneys that eventually lead to a loss of nephron function, further increases in arterial pressure, and severe renal injury in some cases. Although there are many unanswered questions about the mechanisms of obesity hypertension and renal disease, this is one of the most promising areas for future research, especially in view of the growing, worldwide “epidemic” of obesity.

Cicletanine has been shown to enhance natriuresis, thereby countering at least one of the hypertensive effects of obesity cited above (Garay R. P. et al. 1995 Eur J Pharmacol 274:175-180).

### Triglycerides

Reported results from human trials (Tarrade T. & Guinot P. 1988 Drugs Exp Clin Res 14:205-14) include an account of favorable effects upon triglyceride levels in patients receiving higher (150-200 mg/day) of cicletanine. Average triglyceride levels fell from 128 to 104 mg/dl over 12 months.

### HDL Cholesterol

From a study (in Dahl salt-sensitive rats with salt-induced hypertension) reported in 1997, cicletanine treatment significantly decreased low-density lipoprotein (LDL) cholesterol and increased high-density lipoprotein (HDL) cholesterol (Uehara Y. et al. 1997 Blood Press 3:180-7).

### Blood Pressure

Cicletanine is an effective treatment for hypertension (high blood pressure), as cited in numerous articles (see above) and is approved for the treatment of hypertension in several European countries. Cicletanine has been demonstrated as effective both as a monotherapy (Tarrade T. & Guinot P. 1988 Drugs Exp Clin Res 14:205-14) and in combination with other antihypertensive drugs (Tarrade T. et al. 1989 Arch Mal Coeur Vaiss 82 Spec No 4:103-8).

### Fasting Glucose

Fasting glucose is used to assess glucose tolerance. Cicletanine exhibits either a neutral or healthy effect on glucose tolerance. Even at lower doses (50-100 mg per day), cicletanine therapy results in maintained or improved levels of glucose tolerance (Tarrade T. & Guinot P. 1988 Drugs Exp Clin Res 14:205-14). At higher doses (150-200 mg per day; still within the therapeutic/safety range), the positive effect of cicletanine on glucose tolerance becomes more pronounced (Witchitz S. & Gryner S. 1989 Arch Mal Coeur Vaiss 82 Spec No 4:145-9). These positive or neutral effects of cicletanine are in contrast to other antihypertensives, particularly diuretics and beta blockers, which tend to have a deleterious effects upon glucose tolerance and plasma lipids (Brook R. D. 2000 Curr Hypertens Rep 2:370-7).

This favorable comparison of cicletanine with conventional diuretics (per glucose and lipid metabolism) underscores the promise of cicletanine as a component of combination therapy with OADs and lipid-lowering agents, as it should yield distinctive advantages in comparison with the same drugs administered individually.

### EXAMPLES

The persons skilled in the pertinent arts are fully enabled to select a relevant test model to optimize the hereinbefore and hereinafter indicated therapeutic indications. Representative studies are carried out with a combination of cicletanine and a second agent (e.g., antihypertensive agent such as calcium channel blockers, ACE inhibitors, angiotensin II receptor antagonists, etc.) applying the following methodology. Various animal models of diabetes and hypertensive disease are used to evaluate the combination therapy of the present invention. These models include inter alia:
1) an experimental rat model of diabetic nephropathy (uninephrectomized streptozotocin-induced diabetic rats) disclosed by Villa et al. (Am J Hypertens 1997 10:202-8);
2) a rat model exhibiting diabetic hypertension with renal impairment disclosed by Kohzuki et al. (Am J Hypertens 2000 13:298-306 and J Hypertens 1999 17:695-700);
3) a rat model of hypertension in Dahl-S rats fed a high-salt (4% NaCl) diet disclosed by Uehara Y. et al. (J Hypertens 1991 9:719-28);
4) a Sabra rat model of salt-susceptibility previously developed by Prof. Ben-Ishay from the Hebrew University in Jerusalem, which has been transferred to the Rat Genome Center in Ashkelon;
5) a Cohen-Rosenthal Diabetic (Non-Insulin-Dependent) Hypertensive (CRDH) Rat Model for study of diabetic retinopathies www.tau.ac.il/medicine/conf2002/M/M-11.doc;
6) the BB rat (insulin-dependent diabetes mellitus), FHH rat (Fawn hooded hypertensive, ESRD model), GH rat (genetically hypertensive rat), GK rat (noninsulin-dependent diabetes mellitus, ESRD model), SHR (spontaneously hypertensive rat), SR/MCW (salt resistant), SS/MCW (salt sensitive, syndrome-X model) lgr.mcw.edu/lgr_overview.html;
7) a mild hyperglycemic effect of pregnancy on the offspring of type I diabetes can be studied with a rat model established using streptozotocin-induced diabetic pregnant rats transplanted with a controlled number of islets of Langerhans;
8) Zucker diabetic fatty rat (type II);
9) transgenic mice overexpressing the rate-limiting enzyme for hexosamine synthesis, glutamine: F6P amidotransferase (GFA), which results in hyperinsulinemia and insulin resistance (model of type II NIDDM);
10) a two kidney, one clipped rat model of hypertension in STZ-induced diabetes in SD rats;
11) a spontaneously diabetic rat with polyuria, polydipsia, and mild obesity developed by selective breeding (Tokushima Research Institute; Otsuka Pharmaceutical, Tokushima, Japan) and named OLETF. The characteristic features of OLETF rats are 1) late onset of hyperglycemia (after 18 wk of age); 2) a chronic course of disease; 3) mild obesity; 4) inheritance by males; 5) hyperplastic foci of pancreatic islets; and 6) renal complication (Kawano et al. 1992 Diabetes 41:1422-1428); and
12) a spontaneously hypertensive rat (SHR); Taconic Farms, Germantown, N.Y. (Tac:N(SHR)fBR), as disclosed in U.S. Pat. No. 6,395,728.

Of course other animal models and human clinical trials can be employed in accordance with the methodology set forth below.

A radiotelemetric device (Data Sciences International, Inc., St. Paul, Minn.) is implanted into the lower abdominal aorta of all test animals. Test animals are allowed to recover from the surgical implantation procedure for at least 2 weeks prior to the initiation of the experiments. The radiotransmitter is fastened ventrally to the musculature of the inner abdominal wall with a silk suture to prevent movement. Cardiovascular parameters are continuously monitored via the radiotransmitter and transmitted to a receiver where the digitized signal is then collected and stored using a computerized data acquisition system. Blood pressure (mean arterial, systolic and diastolic pressure) and heart rate are monitored in conscious, freely moving and undisturbed animals in their home cages. The arterial blood pressure and heart rate are measured every 10 minutes for 10 seconds and recorded. Data reported for each rat represent the mean values averaged over a 24-hour period and are made up of the 144-10 minute samples collected each day. The baseline values for blood pressure and heart rate consist of the average of three consecutive 24-hour readings taken prior to initiating the drug treatments. All rats are individually housed in a temperature and humidity controlled room and are maintained on a 12 hour light/dark cycle.

In addition to the cardiovascular parameters, determinations of body weight, insulin, blood glucose, urinary thromboxane/PGI₂ ratio (Hishinuma et al. 2001 Prostaglandins, Leukotrienes and Essential Fatty Acids 65:191-196), blood lipids, plasma creatinine, urinary albumin excretion, also are recorded in all rats. Since all treatments are administered in the drinking water, water consumption is measured five times per week. Doses of cicletanine and the second agent (e.g., antihypertensive agents such as calcium channel blockers, ACE inhibitors, angiotensin II receptor antagonists, OADs, or lipid-lowering agents) for individual rats are then calculated based on water consumption for each rat, the concentration of drug substance in the drinking water, and individual body weights. All drug solutions in the drinking water are made up fresh every three to four days.

Upon completion of the 6 week treatment, rats are anesthetized and the heart and kidneys are rapidly removed. After separation and removal of the atrial appendages, left ventricle and left plus right ventricle (total) are weighed and recorded. Left ventricular and total ventricular mass are then normalized to body weight and reported. All values reported for blood pressure and cardiac mass represent the group mean h SEM. The kidneys are dissected for morphological investigation of glomerulosclerosis, renal tubular damage and intrarenal arterial injury.

Cicletanine and the second agent (e.g., calcium channel blockers, ACE inhibitors, angiotensin II receptor antagonists, oral anti-diabetics, oral lipid-lowering agents, etc.) are administered via the drinking water either alone or in combination to rats from beginning at 18 weeks of age and continued for 6 weeks. Based on a factorial design, seven (7) treatment groups are used to evaluate the effects of combination therapy on the above-mentioned indices of hypertension, diabetes and nephropathies. Treatment groups consist of:
1) high dose cicletanine alone in drinking water (in the concentration of about 250-1000 mg/liter);
2) high dose of the second agent alone in drinking water (in a concentration of about 100-500 mg/liter);
3) low dose cicletanine (10-250 mg/liter)+low dose the second agent (1-100 mg/liter);
4) high dose cicletanine+high dose the second agent;
5) high dose cicletanine+low dose the second agent;
6) low dose cicletanine+high dose the second agent; and
7) vehicle control group on regular drinking water.

Thus, 4 groups of rats receive combination therapy. The relative dosages of cicletanine and the second agent can be varied by the skilled practitioner depending on the known pharmacologic actions of the selected drugs. Accordingly, the high and low dosages indicated are provided here only as examples and are not limiting on the dosages that may be selected and tested.

Representative studies are carried out with a combination of cicletanine and other agents, in particular, calcium channel blockers, ACE inhibitors and angiotensin II receptor antagonists, oral anti-diabetics, or lipid-lowering agents. Diabetic renal disease is the leading cause of end-stage renal diseases. Hypertension is a major determinant of the rate of progression of diabetic diseases, especially diabetic nephropathy. It is known that a reduction of blood pressure may slow the reduction of diabetic nephropathy and proteinuria in diabetic patients, however dependent on the kind of antihypertensive administered. In diabetic rat models, the presence of hypertension is an important determinant of renal injury, manifesting in functional changes such as albuminuria and in ultrastructural injury, as detailed in the studies cited above. Accordingly, the use of these animal models are well-applied in the art and suitable for evaluating effects of drugs on the development of diabetic renal diseases. There is a strong need to achieve a significant increase of the survival rate by treatment of hypertension in diabetes especially in non-insulin dependent diabetes mellitus (NIDDM). It is known that calcium channel blockers are not considered as first line antihypertensives e.g., in NIDDM treatment. Though some kind of reduction of blood pressure may be achieved with calcium channel blockers, they may not be indicated for the treatment of renal disorders associated with diabetes.

Diabetes is induced in hypertensive rats aged about 6 to 8 weeks weighing about 250 to 300 g by treatment e.g. with streptozotocin. The drugs are administered by twice daily average. Untreated diabetic hypertensive rats are used as control group (group 1). Other groups of diabetic hypertensive rats are treated with 40 mg/kg of cicletanine (group 2), with high dose of the second agent (group 3) and with a combination of 25 mg/kg of cicletanine and low dose of the second agent (group 4). On a regular basis, besides other parameters the survival rate after 21 weeks of treatment is monitored. In week 21 of the study, survival rates are determined. As discussed above, the dosages can be modified by the skilled practitioner without departing from the scope of the above studies.

The particularly beneficial effect on glycemic control provided by the treatment of the invention is indicated to be a synergistic effect relative to the control expected for the sum of the effects of the individual active agents.

Glycemic control may be characterized using conventional methods, for example by measurement of a typically used index of glycemic control such as fasting plasma glucose or glycosylated hemoglobin (Hb Alc). Such indices are determined using standard methodology, for example those described in: Tuescher A, Richterich, P., Schweiz. Med. Wschr. 101 (1971), 345 and 390 and Frank P., ‘Monitoring the Diabetic Patent with Glycosolated Hemoglobin Measurements’, Clinical Products 1988.

In a preferred aspect, the dosage level of each of the active agents when used in accordance with the treatment of the invention will be less than would have been required from a purely additive effect upon glycemic control.

There is also an indication that the treatment of the invention will effect an improvement, relative to the individual agents, in the levels of advanced glycosylation end products (AGEs), leptin and serum lipids including total cholesterol, HDL-cholesterol, LDL-cholesterol including improvements in the ratios thereof, in particular an improvement in serum lipids including total cholesterol, HDL-cholesterol, LDL-cholesterol including improvements in the ratios thereof, as well as an improvement in blood pressure.

To determine the effect of a compound suitable for use in methods and compositions of the invention on glucose and insulin levels, rats are administered a combination of cicletanine with an oral antidiabetic, after being experimentally induced with type I diabetes, and their urine and blood glucose and insulin levels are determined.

Male Sprague-Dawley (Charles River Laboratories, Montreal, Canada) rats weighing approximately 200 g are randomly separated into control and experimental groups. All experimental animals are given an intravenous injection of 0.1 M citrate buffered streptozotocin (pH 4.5) at a dosage of 65 mg/kg of body weight to induce diabetes mellitus. All control animals receive an intravenous injection of 0.1 M citrate buffer (pH 4.5) alone.

One experimental group of rats also receives daily doses of cicletanine. A second experimental group receives daily sub-therapeutic doses of an oral antidiabetic or lipid-lowering agent. A third experimental group receives both daily doses of cicletanine and a daily sub-therapeutic dose of an oral antidiabetic or lipid-lowering agent.

All animals are fed rat chow and water ad libitum. Plasma glucose levels are done using the Infinity Glucose Reagent® (Sigma Diagnostics, St. Louis, Mo.).

The experimental group of rats that receive daily doses of both daily doses of cicletanine and a daily dose of an oral antidiabetic or lipid-lowering agent show reduced levels of glucose and insulin in blood and urine samples when compared with the group of rats that receive daily sub-therapeutic doses of the oral antidiabetic or lipid-lowering agent without receiving daily doses of cicletanine.

To determine the effect of a composition suitable for use in methods of the invention on glucose and insulin levels, as well as increases in systolic blood pressure, rats having type II diabetes are administered cicletanine, either alone or in combination with sucrose and/or an oral antidiabetic agent, and their systolic blood pressure, urine and blood glucose and insulin levels are determined. Acarbose is known to reduce blood pressure in sucrose induced hypertension in rats (Madar Z et al. Isr J Med Sci 33:153-159).

As described by Madar et al. (Isr J Med Sci 33:153-159), a high sucrose or fructose diet for a prolonged period is one technique used to induce Type II diabetes, specifically hypertension associated with hyperglycemia and hyperinsulinemia in animals.

Male Sprague-Dawley (Charles River Laboratories, Montreal, Canada) rats weighing approximately 200 g are randomly separated into the following groups with each group having 5 animals:

a) The control group that was fed a normal diet and provided with drinking water.

b) The sucrose group that was fed 35% sucrose (35 g sucrose/100 ml of drinking water/day) with an average intake of 150 ml/rat/day.

c) The sucrose+cicletanine group that was fed sucrose as stated in (b) above and cicletanine.

d) The sucrose+OAD group that was fed sucrose as stated in (b) above and administered a therapeutic dose of an OAD.

e) The sucrose+cicletanine+OAD group that was fed sucrose as stated in (b) above, cicletanine, and administered a therapeutic dose of an OAD.

f) The sucrose+cicletanine+OAD group that was fed sucrose as stated in (b) above, cicletanine, and administered subthreshold (subtherapeutic) dose of an OAD.

g) The sucrose+OAD group that was fed sucrose as stated in (b) above and a subthreshold (subtherapeutic) dose of an OAD.

Total duration of the study is 16 weeks. Plasma insulin levels are measured using Rat Insulin RIA Kit (Linco Research Inc., St. Charles, Mo.). Plasma glucose levels are done using the Infinity Glucose Reagent® (Sigma Diagnostics, St. Louis, Mo.). Blood pressure is measured using the tail cuff method (see, Madar et al. Isr J Med Sci 33:153-159).

The results of this study show that when rats are treated with a combination of cicletanine and a therapeutic dose of an OAD a decrease in systolic pressure is significantly greater when compared to rats treated with cicletanine or an OAD alone.

It is the object of this invention to provide a pharmaceutical combination composition, e.g. for the treatment or prevention of a condition or disease selected from the group consisting of hypertension, (acute and chronic) congestive heart failure, left ventricular dysfunction and hypertrophic cardiomyopathy, diabetic cardiac myopathy, supraventricular and ventricular arrhythmias, atrial fibrillation or atrial flutter, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hyperaldosteronism, primary and pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, Raynaud's disease, luminal hyperplasia, cognitive dysfunction (such as Alzheimer's), and stroke, comprising (i) a prostacyclin inducer and (ii) a second agent, preferably an antihypertensive agent, such as calcium channel blocker, an ACE inhibitor or an angiotensin II receptor antagonist, an oral antidiabetic agent, such as a sulfonurea, a biguanide, an alpha-glucosidase inhibitor, a triazolidinedione and a meglitinides, or a lipid-lowering agent.

In this composition, components (i) and (ii) can be obtained and administered together, one after the other or separately in one combined unit dose form or in two separate unit dose forms. The unit dose form may also be a fixed combination.

The determination of the dose of the active ingredients necessary to achieve the desired therapeutic effect is within the skill of those who practice in the art. The dose depends on the warm-blooded animal species, the age and the individual condition and on the manner of administration. In one preferred embodiment, an approximate daily dosage of cicletanine in the case of oral administration is about 10-500 mg/kg/day and more preferably about 30-100 mg/kg/day.

The following example illustrates an oral formulation of one embodiment of the combination invention described above; however, it is not intended to limit its extent in any manner.

An example of a formulation of an oral tablet containing cicletanine and a second agent, such as an antihypertensive, anti-diabetic, or a lipid-lowering agent is as follows. Tablets are formed by roller compaction (no breakline), 200 mg cicletanine+5 mg second agent, with pharmacologically acceptable excipients selected from the group consisting of Avicel PH 102 (filler), PVPP-XL (disintegrant), Aerosil 200 (glidant), and magnesium-stearate (lubricant). Alternatively, an oral tablet containing cicletanine and a second agent may be prepared by wet-granulation followed by compression in a high-speed rotary tablet press, followed by film-coating.

While a number of preferred embodiments of the invention and variations thereof have been described in detail, other modifications and methods of using the disclosed therapeutic combinations will be apparent to those of skill in the art. Accordingly, it should be understood that various applications, modifications, and substitutions may be made of equivalents without departing from the spirit of the invention or the scope of the claims. Further, it should be understood that the invention is not limited to the embodiments set forth herein for purposes of exemplification, but is to be defined only by a fair reading of the appended claims, including the full range of equivalency to which each element thereof is entitled.

All of the references cited herein are incorporated in their entirety by reference thereto.

What is claimed is:

1. An oral formulation, comprising a therapeutically effective amount of cicletanine in combination with a second agent that lowers blood glucose.

2. The oral formulation of claim 1, wherein said first agent comprises a racemic mixture of a (−) and a (+) enantiomers of cicletanine.

3. The oral formulation of claim 1, wherein cicletanine is a (−) enantiomer.

4. The oral formulation of claim 1, wherein cicletanine is a (+) enantiomer.

5. The oral formulation of claim 1, wherein said second agent is selected from the group consisting of sulfonureas, biguanines, alpha-glucosidase inhibitors, triazolidinediones and meglitinides.

6. The oral formulation of claim 5, wherein said second agent is a sulfonurea selected from the group consisting of glimel, glibenclamide; chlorpropamide, tolbutamide, melizide, glipizide and gliclazide.

7. The oral formulation of claim 5, wherein said second agent is a biguanine selected from the group consisting of metformin and diaformin.

8. The oral formulation of claim 5, wherein said second agent is an alpha-glucosidase inhibitor selected from the group consisting of: voglibose; acarbose and miglitol.

9. The oral formulation of claim 5, wherein said second agent is a thiazolidinedione selected from the group consisting of: pioglitazone, rosiglitazone and troglitazone.

10. The oral formulation of claim 5, wherein said second agent is a meglitinide selected from the group consisting of repaglinide and nateglinide.

11. The oral formulation of claim 1, wherein said second agent is a peroxisome proliferator-activated receptor (PPAR) agonist.

12. An oral formulation, comprising a therapeutically effective amount of cicletanine in combination with a second agent that improves a patient's lipid profile.

13. The oral formulation of claim 12, wherein improving said patient's lipid profile comprises at least one change selected from the group consisting of lowering total blood cholesterol, lowering LDL cholesterol, lowering blood triglycerides and raising HDL cholesterol.

14. The oral formulation of claim 12, wherein said first agent comprises a (−) and a (+) enantiomers of cicletanine.

15. The oral formulation of claim 12, wherein cicletanine is a (−) enantiomer.

16. The oral formulation of claim 12, wherein cicletanine is a (+) enantiomer.

17. The oral formulation of claim 12, wherein said second agent is selected from the group consisting of: cholestyramine, colestipol, lovastatin, pravastatin, simvastatin, gemfibrozil, clofibrate, nicotinic acid and probucol.

18. The oral formulation of claim 12, wherein said second agent is a PPAR agonist.

19. A method for treating and/or preventing complications of diabetes or metabolic syndrome in a mammal, comprising administering an oral formulation comprising a therapeutically effective amount of cicletanine and a blood glucose lowering amount of a second agent.

20. The method of claim 19, wherein said second agent is selected from the group consisting of sulfonureas, biguanines, alpha-glucosidase inhibitors, triazolidinediones and meglitinides.

21. The method of claim 20, wherein said second agent is a sulfonurea selected from the group consisting of glimel, glibenclamide; chlorpropamide, tolbutamide, melizide, glipizide and gliclazide.

22. The method of claim 20, wherein said second agent is a biguanine selected from the group consisting of metformin and diaformin.

23. The method of claim 20, wherein said second agent is an alpha-glucosidase inhibitor selected from the group consisting of: voglibose; acarbose and miglitol.

24. The method of claim 20, wherein said second agent is a thiazolidinedione selected from the group consisting of: pioglitazone, rosiglitazone and troglitazone.

25. The method of claim 20, wherein said second agent is meglitinide selected from the group consisting of repaglinide and nateglinide.

26. The method of claim 19, wherein said second agent is a PPAR agonist.

27. The method of claim 19, wherein said complications are selected from the group consisting of retinopathy, neuropathy, nephropathy, microalbuminuria, claudication, macular degeneration, and erectile dysfunction.

28. The method of claim 19, wherein said therapeutically effective amount of cicletanine is sufficient to mitigate a side effect of said second agent.

29. The method of claim 19, wherein said therapeutically effective amount of cicletanine is sufficient to enhance tissue sensitivity to insulin.

30. The method of claim 19, wherein said therapeutically effective amount of cicletanine and said blood glucose lowering amount of said second agent are sufficient to produce a synergistic glucose lowering effect.

31. The method of claim 19, wherein cicletanine comprises a racemic mixture of a (−) and a (+) enantiomers.

32. The method of claim 19, wherein cicletanine is a (−) enantiomer.

33. The method of claim 19, wherein cicletanine is a (+) enantiomer.

34. A method for treating and/or preventing a condition associated with elevated cholesterol in a mammal, comprising administering an oral formulation comprising a therapeutically effective amount of cicletanine and a lipid lowering amount of a second agent.

35. The method of claim 34, wherein said second agent is selected from the group consisting of: cholestyramine, colestipol, lovastatin, pravastatin, simvastatin, gemfibrozil, clofibrate, nicotinic acid and probucol.

36. The method of claim 34, wherein said second agent is an HMG-CoA reductase inhibitor.

37. The method of claim 34, wherein said condition is selected from the group consisting of atherosclerosis, hypertension, retinopathy, neuropathy, nephropathy, microalbuminuria, claudication, macular degeneration, and erectile dysfunction.

38. The method of claim 34, wherein cicletanine comprises a racemic mixture of a (−) and a (+) enantiomers.

39. The method of claim 34, wherein cicletanine is a (−) enantiomer.

40. The method of claim 34, wherein cicletanine is a (+) enantiomer.

41. The method of claim 34, wherein said second agent is a PPAR agonist.

42. A method for treating and/or preventing diabetes or metabolic syndrome comprising administering to a patient in need thereof a therapeutically effective amount of cicletanine, wherein said therapeutically effective amount is sufficient to exert at least two actions selected from the group consisting of lowering blood pressure, decreasing platelet aggregation, lowering blood glucose, lowering total blood cholesterol, lowering LDL cholesterol, lowering blood triglycerides, raising HDL cholesterol, PKC inhibition, and reducing vascular complications associated with diabetes and/or metabolic syndrome.
